(19) United States
(12) Patent Application Publication
(10) Pub. No.: US 2025/0186627 A1
(43) Pub. Date: Jun. 12, 2025

(54) RADIOLABELED COMPOUNDS TARGETING THE PROSTATE-SPECIFIC MEMBRANE ANTIGEN

(71) Applicant: PROVINCIAL HEALTH SERVICES AUTHORITY, Vancouver (CA)

(72) Inventors: Kuo-Shyan LIN, Richmond (CA); François BÉNARD, Vancouver (CA); Chengcheng ZHANG, Richmond (CA); Zhengxing ZHANG, Langley (CA)

(21) Appl. No.: 18/842,681

(22) PCT Filed: Mar. 3, 2023
(86) PCT No.: PCT/CA2023/050280

§ 371 Date: Aug. 29, 2024

Related U.S. Application Data
(60) Provisional application No. 63/316,595, filed on Mar. 4, 2022.

(51) Int. Cl. A61K 51/04; C07B 59/00

(52) U.S. Cl. CPC A61K 51/0406; A61K 51/0497; C07B 59/001; A61K 2121/00; C07B 2200/05

(57) ABSTRACT

A compound comprising a prostate specific membrane antigen (PSMA)-targeting moiety of the following formula (I) or of a salt or a solvate thereof. R¹ is —R^{1a}R^{1b}— wherein R^{1a} is absent, —CH₂—, —O—, or —S—, and R^{1b} is —CH₂— or —CHF—. R² is —(CH₂)₃—O—, —(CH₂)₃—, —(CH₂)₄—, —CH₂—O—(CH₂)₂—, or —CH₂—S—(CH₂)₂. R³ is ethylene fused to a tricyclic fused ring. L¹, L², L³, L⁴, L^{5b} L^{5c}, L⁶ and L⁷ are linkages (e.g. peptide bonds). X^{br} is a branching atom. R^{rad} is a radiometal chelator. R^{alb} is an albumin binder. n1, n2, n3, n4, and n5 are integers. When the PSMA-targeting moiety is linked to a radiolabeling group, the compound may be used as an imaging agent or therapeutic agent for PSMA-expressing diseases/conditions.

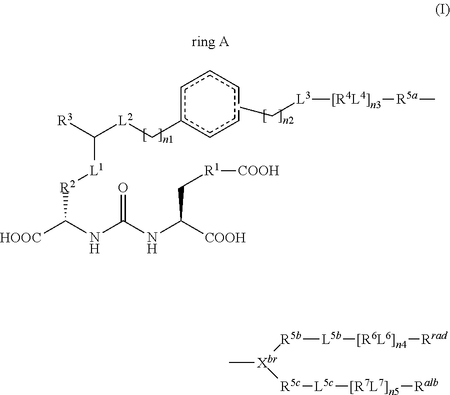

### CROSS-REFERENCE TO RELATED APPLICATIONS

This application claims priority to U.S. Provisional Application No. 63/316,595, filed Mar. 4, 2022, the disclosure of which is herein incorporated by reference in its entirety for all purposes.

### FIELD OF INVENTION

The present invention relates to radiolabelled compounds for treatment of diseases or conditions characterized by expression of prostate-specific membrane antigen, particularly compounds with low uptake in salivary glands and/or kidneys.

### BACKGROUND OF THE INVENTION

Prostate-specific membrane antigen (PSMA) is a transmembrane protein that catalyzes the hydrolysis of N-acetyl-aspartylglutamate to glutamate and N-acetylaspartate. PSMA is selectively overexpressed in certain diseases and conditions compared to most normal tissues. For example, PSMA is overexpressed up to 1,000-fold in prostate tumors and metastases. Due to its pathological expression pattern, various radiolabeled PSMA-targeting constructs have been designed and evaluated for imaging of PSMA-expressing tissues and/or for therapy of diseases or conditions characterized by PSMA expression.

A number of radiolabeled PSMA-targeting derivatives of lysine-urea-glutamate (Lys-ureido-Glu) have been developed, including ¹⁸F-DCFBC, ¹⁸F-DCFPyL, ⁶⁸Ga-PSMA-HBED-CC, ⁶⁸Ga-PSMA-617, ⁶⁸Ga-PSMA I & T (see FIG. 1) as well as versions of the foregoing labelled with alpha emitters (such as ²²⁵Ac) or beta emitters (such as ¹⁷⁷Lu or ⁹⁰Y).

In clinical trials, PSMA-617 radiolabeled with therapeutic radionuclides, such as ¹⁷⁷Lu and ²²⁵Ac, has shown promise as an effective systemic treatment for metastatic castration resistant prostate cancer (mCRPC). However, dry mouth (xerostomia), altered taste and adverse renal events are common side effects of this treatment, due to high salivary gland and kidney accumulation of the radiotracer (Hofman et al., 2018 The Lancet 16(6):825-833; Rathke et al. 2019 Eur J Nucl Med Mol Imaging 46(1):139-147; Sathekge et al. 2019 Eur J Nucl Med Mol Imaging 46(1):129-138). Radiotracer accumulation in the kidneys and salivary gland is therefore a limiting factor that reduces the maximal cumulative administered activity that can be safely given to patients, which limits the potential therapeutic effectiveness of Lys-urea-Glu based radiopharmaceuticals (Violet et al. 2019 J Nucl Med. 60(4):517-523). There is therefore a need for new radiolabeled PSMA-targeting compounds, particularly compounds that have low accumulation in the salivary glands and/or kidneys.

No admission is necessarily intended, nor should it be construed, that any of the preceding information constitutes prior art against the present invention.

### SUMMARY OF THE INVENTION

This disclosure relates to PSMA-targeting compounds that bind PSMA using a Lys-ureido-Glu moiety or derivative moiety thereof, a radiometal chelator, and an albumin binder. The positions of both the radiometal chelator and the albumin binder relative to the PSMA-binding moiety result in novel compounds with useful delivery of radiation to tumor site(s) and improved side effects (e.g. low uptake in kidneys and/or salivary gland). Without wishing to be bound by theory, the disclosed compounds may minimize structural hinderance.

### BRIEF DESCRIPTION OF THE DRAWINGS

The features of the invention will become apparent from the following description in which reference is made to the appended drawings wherein:

FIG. 1 shows examples of prior art PSMA-targeting compounds for prostate cancer imaging.

FIG. 2A shows representative in vitro competitive PSMA binding assay curves using LNCaP cells with CCZ02009 (left panel) and HTK03170 (right panel). FIG. 2B shows PET images of ⁶⁸Ga-CCZ02009 in NRG-mice bearing LNCaP tumors at 1 (left panel) and 3 h p.i. (right panel).

FIG. 3 shows representative in vitro competitive PSMA binding assay curves using LNCaP cells with CCZ02060 (left panel) and CCZ02059 (right panel).

FIG. 4 shows SPECT/CT images of ¹⁷⁷Lu-CCZ02017 in NRG-mice bearing LNCaP tumors at 3, 24, 72, 144 and 240 h p.i.

FIG. 5 shows a representative in vitro competitive PSMA binding assay curve using LNCaP cells with CCZ02008.

FIG. 6 shows a representative in vitro competitive PSMA binding assay curve using LNCaP cells with CCZ02025.

FIG. 7 shows a representative in vitro competitive PSMA binding assay curve using LNCaP cells with CCZ02024.

FIG. 8 shows a representative in vitro competitive PSMA binding assay curve using LNCaP cells with CCZ02015.

FIGS. 9A-9B show representative in vitro competitive PSMA binding assay curves using LNCaP cells with CCZ02012 (FIG. 9A) and CCZ02013 (FIG. 9B).

FIGS. 10A-10B show representative in vitro competitive PSMA binding assay curves using LNCaP cells with CCZ02021 (FIG. 10A) and CCZ02022 (FIG. 10B).

FIG. 11 shows a representative in vitro competitive PSMA binding assay curve using LNCaP cells with CCZ02034.

FIG. 12 shows a representative in vitro competitive PSMA binding assay curve using LNCaP cells with CCZ02005.

FIG. 13 shows a representative in vitro competitive PSMA binding assay curve using LNCaP cells with CCZ02061.

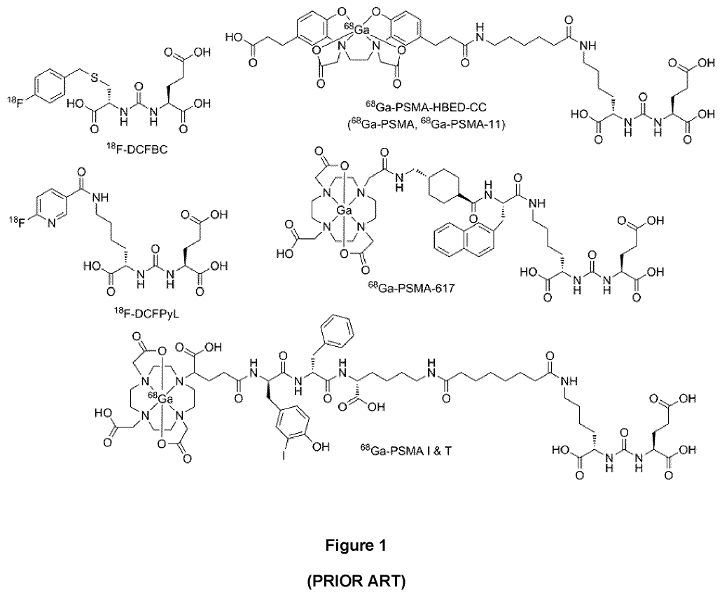

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, etcetera.

As used herein, the terms “treat”, “treatment”, “therapeutic” and the like includes ameliorating symptoms, reducing disease progression, improving prognosis and reducing recurrence.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, salts or pharmaceutically acceptable salts thereof. Unless otherwise specified, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g. protonated) state or may be shown without specifying formal charges. As will be appreciated by the person of skill in the art, the ionization state of certain groups within a compound (e.g. without limitation, CO₂H, PO₃H₂, SO₂H, SO₃H, SO₄H, OPO₃H₂ and the like) is dependent, inter alia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized. Likewise, OSO₃H (i.e. SO₄H) groups, SO₂H groups, SO₃H groups, OPO₃H₂ (i.e. PO₄H₂) groups and PO₃H groups would generally be deprotonated (and negatively charged) at neutral and physiological pH values.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, the expression “Cₙ” where n is an integer (e.g. 1, 2, 3, 4, 5, 6, 7, 8, 9, 10, 11, 12, 13, 14, 15, 16, 17, 18, 19, 20, and the like) or where n is defined as a range of integers (e.g. 1-20, 1-18, 2-15, 3-20, and the like) refers to the number of carbons in a compound, R-group, L-group, or substituent, or refers to the number of carbons plus heteroatoms in a compound, R-group, L-group, or substituent. A range of integers includes all integers in the range; e.g. the range 1-20 includes the integers 1, 2, 3, 4, 5, 6, 7, 8, 9, 10, 11, 12, 13, 14, 15, 16, 17, 18, 19, and 20. Unless otherwise defined, heteroatoms may include any, some or all possible heteroatoms. For example, in some embodiments, the heteroatoms may be selected from N, O, S, P and Se. In some embodiments, the heteroatoms are selected from N, S, or O. Such embodiments are non-limiting. The alternative expression “Cy-Cz”, where y and z are integers (e.g. C₃-C₁₅ and the like), is equivalent to “Cₙ” where n is a range of integers from y to z.

The terms “alkyl”, “alkylenyl”, “alkenylenyl”, and “alkynylenyl” have their usual meanings in organic chemistry. For example, an “alkyenylenyl” has at least one carbon-carbon double bond, and may have any number of carbon-carbon single bonds. Similarly, an “alkynylenyl” has at least one carbon-carbon triple bond, and may have any number of carbon-carbon single bonds. The expressions “alkylenyl, alkenylenyl and/or alkynylenyl” and “alkylenyl, alkenylenyl or alkynylenyl” are intended to be equivalent and each includes hydrocarbon chains that can have any reasonable number or combination of carbon-carbon single bonds, double bonds, and triple bonds. These hydrocarbon chains can be linear, branched, cyclic, or any combination of linear and branched, linear and cyclic, cyclic and branched, branched and cyclic, or linear, branched and cyclic. Cyclic hydrocarbons may be nonaromatic, partially aromatic, or aromatic. Unless otherwise specified, the term “cyclic” includes single rings, multiple non-fused rings, fused rings, bridged rings, and combinations thereof.

The expression “wherein any carbon . . . is optionally independently replaced by N, S, or O” and other similar expressions means that the defined hydrocarbon (e.g. “alkyl”, “alkylenyl”, “alkenylenyl”, or “alkynylenyl”) includes zero, one, more than one, or any reasonable combination of two or more heteroatoms selected from N, S, and O. The above expression therefore expands the defined hydrocarbon to additionally encompass heteroalkyls, heteroalkylenyls, heteroalkenylenyls, and heteroalkynylenyls, etc. The person of skill in the art would understand that various combinations of different heteroatoms may be used. The expression “wherein any carbon bonded to two other carbons is optionally independently replaced by N, S, or O” and other similar expressions means that any carbon in the defined hydrocarbon bonded to two other carbons (e.g. the underlined carbon in —C—C—C—), whether those bonds are single, double, or triple bonds, may be a heteroatom, but excludes heteroatoms bonded to other heteroatoms (e.g. excludes —C—N—S—, —S—S—N—, —N—S—C—, and the like).

Various R-groups (e.g. R¹, R², R³, etc.) and L-groups (e.g. L¹, L², L³, etc.) are defined in this disclosure. L-groups generally refer to linkages (e.g. —S—, —NH—C(O)—, —C(O)—NH—, —N(alkyl)-C(O)—, —C(O)—N(alkyl)-, —NH—C(O)—NH—, —NH—C(S)—NH—,

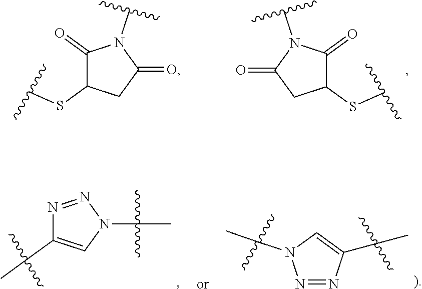

If unspecified, the size of an R-group or L-group is what would be considered reasonable to the person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons in length, subject to the common general knowledge of the person of skill in the art.

Further, but without limitation, if unspecified, the size of a heteroalkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons and heteroatoms in length, subject to the common general knowledge of the person of skill in the art. In the context of the expression “alkyl, alkenyl or alkynyl” and similar expressions, the “alkyl” would be understood to be a saturated alkyl, and the “alkenyl” and the “alkynyl” would be understood to be unsaturated.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

The term “alkylenyl” refers to a divalent analog of an alkyl group. In the context of the expression “alkylenyl, alkenylenyl and/or alkynylenyl”, and similar expressions, the “alkylenyl” would be understood to be a saturated alkylenyl, and the “alkenylenyl” and the “alkynylenyl” would be understood to be unsaturated. The term “heteroalkylenyl” refers to a divalent analog of a heteroalkyl group. The term “heteroalkenylenyl” refers to a divalent analog of a heteroalkenyl group.

The term “heteroalkynylenyl” refers to a divalent analog of a heteroalkynyl group.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, I-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, I-propene-2-yl, 1-butene-1-yl, 1-butene-2-yl, 1-butene-3-yl, 2-butene-1-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. Unless otherwise specified, a C₁-C₂₀ alkynylenyl therefore encompasses, without limitation, all divalent analogs of the above-listed alkynyl groups.

Non-limiting examples of non-aromatic cyclic groups include cylcopropyl, cyclobutyl, cyclopentyl, cyclohexyl, and the like. Non-limiting examples of non-aromatic heterocyclic groups include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. non-limiting examples of C₃-C₂₀ aryl groups include phenyl (Ph), pentalenyl, indenyl, naphthyl and azulenyl. Non-limiting examples of aromatic heterocyclic groups of similar size include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like.

As used herein, the term “substituted” is used as it would normally be understood to a person of skill in the art and generally refers to a compound or chemical entity that has one chemical group replaced with a different chemical group. Unless otherwise specified, a substituted alkyl, alkylenyl, alkenylenyl, or alkynylenyl has one or more hydrogen atom(s) independently replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g. R-group or L-group) may be substituted with any chemical group reasonable to the person of skill in the art. For example, but without limitation, a hydrogen bonded to a carbon or heteroatom (e.g. N) may be substituted with halide (e.g. F, I, Br, Cl), amine, amide, oxo, hydroxyl, thiol, phosphate, phosphonate, sulfate, SO₂H, SO₃H, alkyls, heteroalkyls, aryl, heteroaryl, ketones, carboxaldehyde, carboxylates, carboxamides, nitriles, monohalomethyl, dihalomethyl ortrihalomethyl. In some embodiments, each carbon may be independently substituted or unsubstituted with oxo, hydroxyl, sulfhydryl, amine, amide, urea, halogen, guanidino, carboxylic acid, sulfonic acid, sulfinic acid, or phosphoric acid. In some embodiments, the amide substituent is —C(O)—NH₂.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”. The expression “optionally independently substituted” means that each location may be substituted or may not be substituted, and when substituted each substituent may be the same or different.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The compounds disclosed herein may be synthesized (at least in part) using peptide synthesis methods. Each amino acid residue in a peptide or peptidic region has both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, amino acid residues may have the formula —N(R^{a})—R^{b}—C(O)—, where R^{a} and R^{b} are R-groups. R^{a} will typically be hydrogen or methyl (or a different alkyl). The amino acid residues of a peptide may comprise typical peptide (amide) bonds and may further comprise bonds between side chain functional groups and the side chain or main chain functional group of another amino acid.

For example, the side chain carboxylate of one amino acid residue (e.g. Asp, Glu, etc.) in the peptide or peptidic region may be bonded to the amine of another amino acid residue (e.g. Dap, Dab, Orn, Lys) in the peptide or peptidic region. Further details are provided below. Unless otherwise indicated, an amino acid may be any amino acid, including proteinogenic and nonproteinogenic amino acids, alpha amino acids, beta amino acids, or any other amino acid. Non-limiting examples of nonproteinogenic amino acids are shown in Table 1 and include: D-amino acids (including without limitation any D-form of the following amino acids), ornithine (Orn), 3-(1-naphtyl)alanine (NaI), 3-(2-naphtyl)alanine (2-NaI), α-aminobutyric acid, norvaline, norleucine (NIe), homonorleucine, beta-(1,2,3-triazol-4-yl)-L-alanine, 1,2,4-triazole-3-alanine, Phe(4-F), Phe(4-Cl), Phe(4-Br), Phe(4-1), Phe(4-NH₂), Phe(4-NO₂), homoarginine (hArg), 2-amino-4-guanidinobutyric acid (Agb), 2-amino-3-guanidinopropionic acid (Agp), β-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl) propanoic acid, 2-amino-3-(anthracen-9-yl)propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), and tranexamic acid. If not specified as an L- or D-amino acid, an amino acid shall be understood to encompass both L- and D-amino acids.

TABLE 1
List of non-limiting examples of non-proteinogenic amino acids.

| | |
|---|---|
| Any D-amino acid of a proteinogenic amino acid | 10-aminodecanoic acid |
| ornithine (Orn) | 2-aminooctanoic acid |
| 3-(1-naphtyl)alanine (Nal) | 2-amino-3-(anthracen-2-yl)propanoic acid |
| 3-(2-naphtyl)alanine (2-Nal) | 2-amino-3-(anthracen-9-yl)propanoic acid |
| α-aminobutyric acid | 2-amino-3-(pyren-1-yl)propanoic acid |
| norvaline | Trp(5-Br), |
| norleucine (Nle) | Trp(5-OCH₃), |
| homonorleucine | Trp(6-F), |
| beta-(1,2,3-triazol-4-yl)-L-alanine | Trp(5-OH) |
| 1,2,4-triazole-3-alanine | Trp(CHO), |
| Phe(4-F), Phe(2-F), Phe(3-F), | 2-aminoadipic acid (2-Aad) |
| Phe(4-Cl), Phe(2-Cl), Phe(3-Cl), | 3-aminoadipic acid (3-Aad) |
| Phe(4-Br), Phe(2-Br), Phe(3-Br), | propargylglycine (Pra) |
| Phe(4-1), Phe(2-1), Phe(2-1), | homopropargylglycine (Hpg) |
| Phe(4-NH₂), Phe(2-NH₂), Phe(3-NH₂), | beta-homopropargylglycine (Bpg) |
| Phe(4-NO₂), Phe(2-NO₂), Phe(2-NO₂), | 2,3-diaminopropionic acid (Dap) |
| homoarginine (hArg) | 2,4-diaminobutyric acid (Dab) |
| 4-(2-aminoethyl)-1-carboxymethyl-piperazine (Acp) | azidolysine (Lys(N₃)) |
| 2-(5′-azidopentyl)alanine, 2-(6′-azidohexyl)alanine | azido-ornithine (Orn(N₃)) |
| 2-amino-4-guanidinobutyric acid (Agb) | amino-4-azidobutanoic acid Dab(N₃) |
| 2-amino-3-guanidinopropionic acid (Agp) | tranexamic acid |
| β-alanine | 4-amino-1-carboxymethyl-piperidine (Pip) |
| 4-aminobutyric acid | NH₂(CH₂)₂O(CH₂)₂C(O)OH |
| 5-aminovaleric acid | NH₂(CH₂)₂[O(CH₂)₂]₂C(O)OH |
| 6-aminohexanoic acid | NH₂(CH₂)₂[O(CH₂)₂]₃C(O)OH |
| 7-aminoheptanoic acid | NH₂(CH₂)₂[O(CH₂)₂]₄C(O)OH |
| 8-aminooctanoic acid | NH₂(CH₂)₂[O(CH₂)₂]₅C(O)OH |
| 9-aminononanoic acid | NH₂(CH₂)₂[O(CH₂)₂]₆C(O)OH |

The wavy line “ [symbol] ” symbol shown through or at the end of a bond in a chemical formula (e.g. in the definitions L¹, R³, L², L³ and R^{alb} of Formula I) is intended to define the group on one side of the wavy line, without modifying the definition of the structure on the opposite side of the wavy line. Where an R-group or L-group is bonded on two or more sides, any atoms shown outside the wavy lines are intended to clarify orientation of the defined group. As such, only the atoms between the two wavy lines constitute the definition of the R-group or L-group. When atoms are not shown outside the wavy lines (e.g. L¹), or for a chemical group shown without wavy lines but does have bonds on multiple sides (e.g. —C(O)NH—, and the like), the chemical group should be read from left to right matching the orientation in the formula that the group relates to; e.g. for formula —R^{a}—R^{b}—R^{c}—, the definition of R^{b} as —C(O)NH— would be incorporated into the formula as —R^{a}—C(O)NH—R⁰— not as —R^{a}—NHC(O)—R⁰—.

In various aspects, there is disclosed a compound wherein the compound has Formula I (as defined below) or wherein the compound is a salt or a solvate of Formula I:

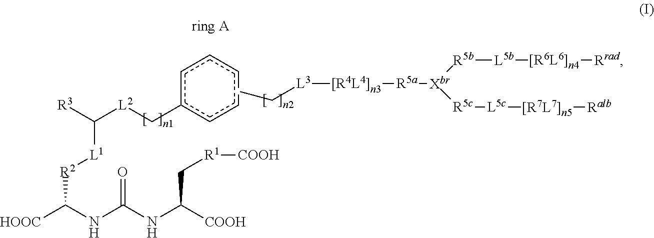

wherein:
R¹ is —R^{1a}R^{1b}—, wherein R^{1a} is absent, —CH₂—, —O—, or —S—, and R^{1b} is —CH₂— or —CHF—;
R² is —(CH₂)₃—O—, —(CH₂)₃—, —(CH₂)₄—, —CH₂—O—(CH₂)₂—, or —CH₂—S—(CH₂)₂—;
L¹ is —S—, —N(R^{L1a})—C(O)—, —C(O)—N(R^{L1a})—, —NH—C(O)—NH—, —NH—C(S)—NH—,

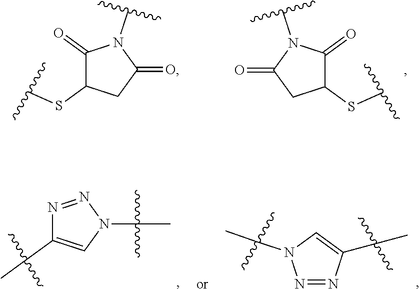

wherein R^{L1a} is H, methyl, ethyl or a benzyl group with 0-4 substituents independently selected from halogen, OMe, or SMe;
R³ is:

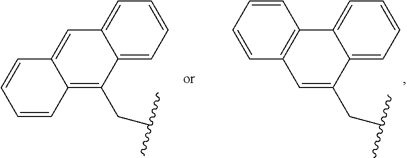 [SMILES: *Cc1c2ccccc2cc2ccccc12 ; *Cc1cc2ccccc2c2ccccc12]

substituted with 0-4 substituents independently selected from C₁-C₄ alkyl, halogen, OMe, SMe, NH₂, NO₂, CN, or OH, and wherein 0-4 ring carbons are replaced with nitrogen;
L² is —S—, —N(R^{L2a})—C(O)—, —C(O)—N(R^{L2a})—, —NH—C(O)—NH—, —NH—C(S)—NH—,

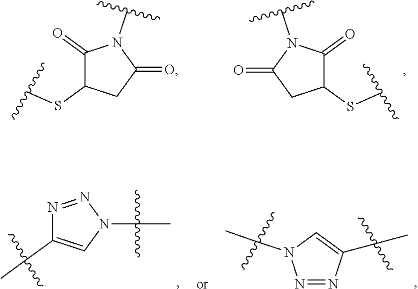

wherein R^{L2a} is H, methyl, or ethyl;
each of n1 and n2 is 0-2;
ring A has 0-3 double bonds, and is bonded at meta or para position;
each of L³, L⁴, L^{5b}, L^{5c}, L⁶, and L⁷ is independently —S—, —N(R^{L3a})—C(O)—, —C(O)—N(R^{L3a})—, —NH—C(O)—NH—, —NH—C(S)—NH—,

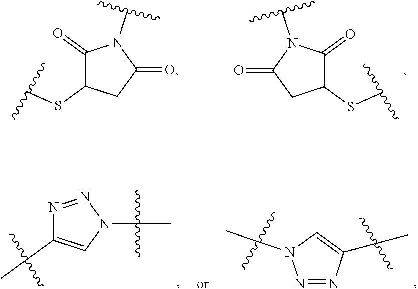

wherein each R^{L3a} is independently H, methyl, or ethyl;
each of n3, n4, and n5 is independently 0-4;
each R⁴ is independently a linear, branched, and/or cyclic Cₙ₆ alkylenyl, alkenylenyl and/or alkynylenyl, wherein each n6 is independently 1-20, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid; and
R^{5a}—X^{br}(R^{5c})—R^{5b} forms a linear, branched, and/or cyclic Cₙ₇ alkylenyl, alkenylenyl and/or alkynylenyl, wherein n7 is 1-20, wherein any carbon bonded to two other carbons is optionally independently replaced by N, S, or O, wherein carbons are optionally independently substituted with oxo, hydroxyl, sulfhydryl, amine, amide, urea, halogen, guanidino, carboxylic acid, sulfonic acid, sulfinic acid, or phosphoric acid, wherein one, two, or three of R^{5a}, R^{5b}, and R^{5c} are optionally absent, wherein X^{br} is C, CH, or N and wherein X^{br} is separated from ring A by at least 4 atoms;
each R⁶ and each R⁷ is independently a linear, branched, and/or cyclic Cₙ₈ alkylenyl, alkenylenyl and/or alkynylenyl, wherein each n8 is independently 1-20, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid;
R^{rad} is a radiometal chelator, optionally bound by a radiometal, wherein R^{rad} is separated from ring A by at least 7 atoms; and
R^{alb} is an albumin binder, wherein the albumin binder is:
   —(CH₂)ₙ₉—CH₃ wherein n9 is 8-20;
   —(CH₂)ₙ₁₀—C(O)OH wherein n10 is 8-20; or

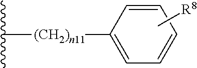

wherein n11 is 1-4 and R⁸ is I, Br, F, Cl, H, OH, OCH₃, NH₂, NO₂, or CH₃; and
R^{alb} is separated from ring A by at least 7 atoms.

In some embodiments, the compound (of Formula I) has Formula II or is a salt or a solvate of Formula II:

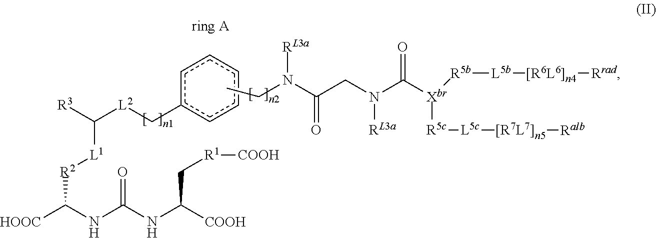

wherein R¹, R², R³, R^{L3a}, R^{5b}, R^{5c}, R⁶, R⁷, R^{rad}, R^{alb}, L¹, L², L^{5b}, L^{5c}, L⁶, L⁷, ring A, n1, n2, n4, and n5 are as defined in Formula I, or as defined in any other embodiment(s) defined herein. In some of these embodiments: R¹ is —CH₂—CH₂— or —CHF—. In some of these embodiments, R² is —(CH₂)₄—. In some of these embodiments, L¹ is —NH—C(O)—. In some of these embodiments, R³ is

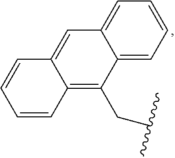 [SMILES: *Cc1c2ccccc2cc2ccccc12]

optionally wherein R³ is

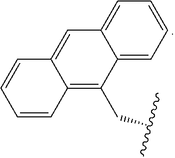

In some of these embodiments, L² is —NHC(O)—. In some of these embodiments, n1 is 0; ring A has 0 double bonds and is bonded at para position, optionally wherein ring A is

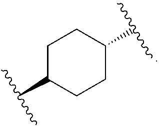

In some of these embodiments, n2 is 0 or 1. In some of these embodiments, each R^{L3a} is H. In some of these embodiments, X^{br} is N or CH. In some of these embodiments: R^{5b} is —(CH₂)₄— and R^{5c} is absent; or R^{5c} is —(CH₂)₄— and R^{5b} is absent. In some of these embodiments, L^{5b} is —NH—C(O)—. In some of these embodiments, L^{5c} is —NH—C(O)—. In some of these embodiments, L^{5b} is —NH—C(O)—. In some of these embodiments, n4 is 0 or 1, and R⁶—when present—is methylene. In some of these embodiments, n5 is 0 or 1, and R⁷—when present—is methylene. In some of these embodiments, L⁶—when present—is —NH—C(O)—. In some of these embodiments, L⁷—when present—is —NH—C(O)—. In some of these embodiments, R^{alb} is

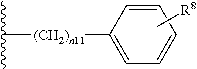

wherein n11 is 3 and R^{a} is OCH₃ or NO₂. In some of these embodiments, R^{rad} is DOTA or a DOTA derivative.

In some embodiments, R¹ is —R^{1a}R^{1b}—, wherein R^{1a} is absent or —CH₂—, and R^{1b} is —CH₂— or —CHF;
R² is —(CH₂)₃—O—, —(CH₂)₃—, —(CH₂)₄—, or —CH₂—O—(CH₂)₂—;
L¹ is —N(R^{L1a})—C(O)—, —C(O)—N(R^{L1a})—, or —NH—C(O)—NH—, wherein R^{L1a} is H or methyl;
R³ is:

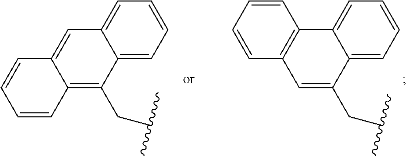 [SMILES: *Cc1c2ccccc2cc2ccccc12 ; *Cc1cc2ccccc2c2ccccc12]

L² is —N(R^{L2a})—C(O)—, —C(O)—N(R^{L2a})—, —NH—C(O)—NH—, wherein R^{L2a} is H, or methyl;
each of n1 and n2 is 0-2;
ring A has 0 bonds, and is bonded at meta or para position;
each of L³, L⁴, L^{5b}, L^{5c}, L⁶, and L⁷ is independently —N(R^{L3a})—C(O)—, —C(O)—N(R^{L3a})—, or —NH—C(O)—NH—, wherein each R^{L3a} is independently H, methyl, or ethyl;
each of n3, n4, and n5 is independently 0-4;
each R⁴ is independently a linear C₁-C₁₀ alkylenyl;
R^{5a}—X^{br}(R^{5c})—R^{5b} forms a linear, branched, and/or cyclic C₁-C₂₀ alkylenyl, alkenylenyl and/or alkynylenyl, wherein any carbon bonded to two other carbons is optionally independently replaced by N, S, or O, wherein carbons are optionally independently substituted with oxo, hydroxyl, sulfhydryl, amine, amide, urea, halogen, guanidino, carboxylic acid, sulfonic acid, sulfinic acid, or phosphoric acid, wherein one, two, or three of R^{5a}, R^{5b}, and R^{5c} are optionally absent, wherein X^{br} is N or CH, and wherein X^{br} is separated from ring A by at least 4 atoms;
each R⁶ and each R⁷ is independently a linear, branched, and/or cyclic C₁-C₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid;
R^{rad} is a radiometal chelator, optionally bound by a radiometal, wherein R^{rad} is separated from ring A by at least 7 atoms; and R^{alb} is:

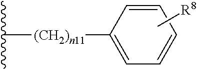

wherein n11 is 1-4 and R⁸ is I, Br, F, Cl, H, OH, OCH₃, NH₂, NO₂, or CH₃.

In another embodiment, R² is —(CH₂)₃—, or —(CH₂)₄—;
L¹ is —N(R^{L1a})—C(O)—, or —C(O)—N(R^{L1a})—, wherein R^{L1a} is H or methyl;
R³ is:

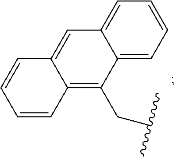 [SMILES: *Cc1c2ccccc2cc2ccccc12]

L² is —N(R^{L2a})—C(O)—, or —C(O)—N(R^{L2a})—, wherein R^{L2a} is H, or methyl;
each of n1 and n2 is 0-1;
each of L³, L⁴, L^{5b}, L^{5c}, L⁶, and L⁷ is independently —N(R^{L3a})—C(O)—, or —C(O)—N(R^{L3a})—, wherein each R^{L3a} is independently H or methyl;
each R⁴ is independently a linear C₁-C₁₀ alkylenyl;
R^{5a}—X^{br}(R^{5c})—R^{5b} forms a linear, branched, and/or cyclic C₁-C₁₀ alkylenyl, wherein any carbon bonded to two other carbons is optionally independently replaced by O, wherein carbons are optionally independently substituted with oxo, hydroxyl, amine, amide, or carboxylic acid, wherein one, two, or three of R^{5a}, R^{5b}, and R^{5c} are optionally absent, wherein X^{br} is N or CH and wherein X^{br} is separated from ring A by at least 4 atoms;
each R⁶ and each R⁷ is independently a linear, branched, and/or cyclic C₁-C₁₀ alkylenyl, wherein any carbon bonded to two other carbons is optionally independently replaced by N, S, or O, and carbons are optionally independently substituted with oxo, hydroxyl, amine, amide, urea, carboxylic acid; R^{alb} is:

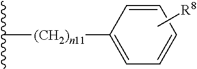

wherein n11 is 1-4 and R⁸ is OCH₃, or NO₂.

In another specific embodiment, L³ is —NHC(O)—. In another specific embodiment, L⁴ is —NHC(O)—. In another specific embodiment, R⁴ is methylene. In another specific embodiment, n3 is 1-4. In another specific embodiment, n3 is 1-2. In another specific embodiment, n3 is 1. In another specific embodiment, n2 is 0-1. In another specific embodiment, n2 is 1. In another specific embodiment, R^{5a} is absent. In another specific embodiment, R^{5a} is methylene.

In some embodiments, R^{1a} is absent. In some embodiments, R^{1a} is —CH₂—. In some embodiments, R^{1a} is —O—. In some embodiments, R^{1a} is —S—.

In some embodiments, R^{1b} is —CH₂—. In some embodiments, R^{1b} is —CHF—.

In some embodiments, R¹ is —CH₂—. In some embodiments, R¹ is —CHF—. In some embodiments, R¹ is —CH₂—CH₂—. In some embodiments, R¹ is —CH₂—CHF—. In some embodiments, R¹ is —O—CH₂—. In some embodiments, R¹ is —O—CHF—. In some embodiments, R¹ is —S—CH₂—. In some embodiments, R¹ is —S—CHF—.

In some embodiments, R² is —(CH₂)₃—O—. In some embodiments, R² is —(CH₂)₃—. In some embodiments, R² is —(CH₂)₄—. In some embodiments, R² is —CH₂—O—(CH₂)₂—. In some embodiments, R² is —CH₂—S—(CH₂)₂—.

In some embodiments, L¹ is —N(R^{L1a})—C(O)— or —C(O)—N(R^{L1a})— wherein R^{L1a} is as defined in Formula I. In some such embodiments, R^{L1a} is H. In other such embodiments, R^{L1a} is methyl. In other such embodiments, R^{L1a} is ethyl. In yet other such embodiments, R^{L1a} is a benzyl group with 0-4 substituents independently selected from halogen, OMe, or SMe. In yet other such embodiments, R^{L1a} is a benzyl group. In some embodiments, L¹ is —N(R^{L1a})—C(O)— or —(O)—N(R^{L1a})— wherein L¹a is H or methyl. In some embodiments, L¹ is —NHC(O)—. In some embodiments, L¹ is —C(O)—NH—.

In some embodiment, R^{5a}—X^{br}(R^{5c})—R^{5b} forms a linear or branched C₁-C₂₀ alkylenyl, wherein any carbon bonded to two other carbons is optionally independently replaced by 0, wherein carbons are optionally independently substituted with oxo, hydroxyl, amine, amide, or carboxylic acid, wherein one, two, or three of R^{5a}, R^{5b}, and R^{5c} are optionally absent, wherein X^{br} is N or CH and wherein X^{br} is separated from ring A by at least 4 atoms. In another specific embodiment, the R^{5a}—X^{br}(R^{5c})—R^{5b} forms a linear or branched C₁-C₁₀ alkylenyl, wherein any carbon bonded to two other carbons is optionally independently replaced by O. In another specific embodiment, 1, 2, 3 or 4 carbons are replaced by O.

In some embodiments, L¹ is —S—. In some embodiments, L¹ is —NH—C(O)—NH—. In some embodiments, L¹ is —NH—C(S)—NH—. In some embodiments, L¹ is

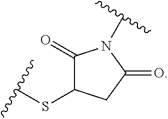 [SMILES: *SC1CC(=O)N(*)C1=O]

In some embodiments, L¹ is

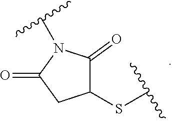 [SMILES: *SC1CC(=O)N(*)C1=O]

In some embodiments, L¹ is

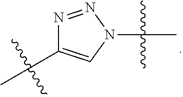 [SMILES: *c1cn(*)nn1]

In some embodiments, L¹ is

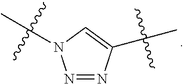 [SMILES: *c1cn(*)nn1]

In some embodiments, R³ is:

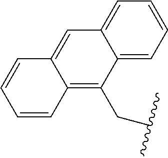 [SMILES: *Cc1c2ccccc2cc2ccccc12]

substituted with 0-4 substituents independently selected from C₁-C₄ alkyl, halogen, OMe, SMe, NH₂, NO₂, CN, or OH, and wherein 0-4 ring carbons are replaced with nitrogen; in some such embodiments, the rings are unsubstituted and contain a single nitrogen. In some embodiments, R³ is:

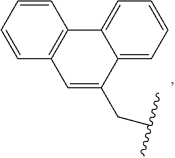 [SMILES: *Cc1cc2ccccc2c2ccccc12]

substituted with 0-4 substituents independently selected from C₁-C₄ alkyl, halogen, OMe, SMe, NH₂, NO₂, CN, or OH, and wherein 0-4 ring carbons are replaced with nitrogen; in some such embodiments, the rings are unsubstituted and contain a single nitrogen. In some embodiments, R³ is:

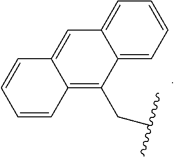 [SMILES: *Cc1c2ccccc2cc2ccccc12]

In some embodiments, R³ is:

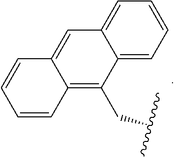

In some embodiments, R³ is:

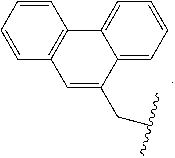 [SMILES: *Cc1cc2ccccc2c2ccccc12]

In some embodiments, R³ is:

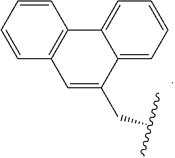

In some embodiments, L² is —N(R^{L2a})—C(O)— or —C(O)—N(R^{L2a})— wherein R^{L2a} is as defined in Formula I. In some such embodiments, R^{L2a} is H. In other such embodiments, R^{L2a} is methyl. In other such embodiments, R^{L2a} is ethyl. In yet other such embodiments, R^{L2a} is a benzyl group with 0-4 substituents independently selected from halogen, OMe, or SMe. In yet other such embodiments, R^{L2a} is a benzyl group. In some embodiments, L² is —N(R^{L2a})—C(O)— or —(O)—N(R^{L2a})— wherein L^{2a} is H or methyl. In some embodiments, L² is —NHC(O)—. In some embodiments, L² is —C(O)—NH—.

In some embodiments, L² is —S—. In some embodiments, L² is —NH—C(O)—NH—. In some embodiments, L² is —NH—C(S)—NH—. In some embodiments, L² is

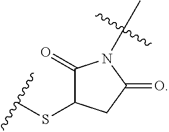 [SMILES: *SC1CC(=O)N(*)C1=O]

In some embodiments, L² is

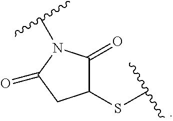 [SMILES: *SC1CC(=O)N(*)C1=O]

In some embodiments, L² is

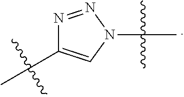 [SMILES: *c1cn(*)nn1]

In some embodiments, L² is

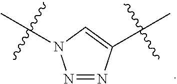 [SMILES: *c1cn(*)nn1]

In some embodiments, n1 is 0. In some embodiments, n1 is 1. In some embodiments n1 is 2.

In some embodiments, ring A has 0 double bonds (i.e. all single bonds). In some embodiments, ring A has 1 double bond. In some embodiments, ring A has 2 double bonds. In some embodiments, ring A has 3 double bonds. In some embodiments, ring A is bonded at meta position. In some embodiments, ring A is bonded at para position. In some embodiments, ring A has 0 double bonds and is bonded at para position. In some embodiments, ring A is

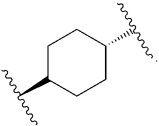

In some embodiments, ring A is

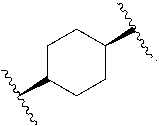

In some embodiments, n2 is 0. In some embodiments, n2 is 1. In some embodiments n2 is 2.

In some embodiments, L³ is —N(R^{L3a})—C(O)— or —C(O)—N(R^{L3a})— wherein R^{L3a} is as defined in Formula I. In some such embodiments, R^{L3a} is H. In other such embodiments, R^{L3a} is methyl. In other such embodiments, R^{L3a} is ethyl. In some embodiments, L³ is —N(R^{L3a})—C(O)— or —(O)—N(R^{L3a})— wherein R^{L3a} is H or methyl. In some embodiments, L³ is —NHC(O)—. In some embodiments, L³ is —C(O)—NH—. In some embodiments, L³ is —S—. In some embodiments, L³ is —NH—C(O)—NH—. In some embodiments, L³ is —NH—C(S)—NH—. In some embodiments, L³ is

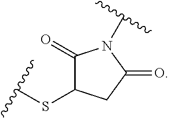 [SMILES: *SC1CC(=O)N(*)C1=O]

In some embodiments, L³ is

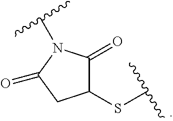 [SMILES: *SC1CC(=O)N(*)C1=O]

In some embodiments, L³ is

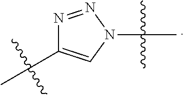 [SMILES: *c1cn(*)nn1]

In some embodiments, L³ is

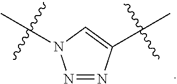 [SMILES: *c1cn(*)nn1]

In some embodiments, R¹ is —CH₂—CH₂— and R² is —(CH₂)₄—. In some such embodiments, L¹ is —NH—C(O)—. In some such embodiments, R³ is

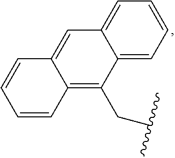 [SMILES: *Cc1c2ccccc2cc2ccccc12]

optionally wherein R³ is

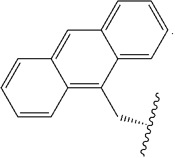

In some such embodiments, L² is —NHC(O)—. In some such embodiments, n1 is 0; ring A has 0 double bonds and is bonded at para position, optionally wherein ring A is

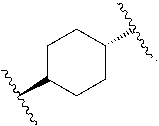

In some such embodiments, n2 is 0 or 1. In some such embodiments, L³ is —NHC(O)—.

In some embodiments, n3 is 0. In some embodiments, n3 is 1. In some embodiments n3 is 2. In some embodiments, n3 is 3. In some embodiments n3 is 4.

In embodiments where n3 is not zero, each R⁴ is independently a linear, branched, and/or cyclic Cₙ₆ alkylenyl, alkenylenyl and/or alkynylenyl, wherein any carbon bonded to two other carbons is optionally independently replaced by N, S, or O, and carbons are optionally independently substituted. In some embodiments, each n6 is independently 1-15 or 1-10. In alternative embodiments, each n6 is independently 1, 2, 3, 4, 5, 6, 7, 8, 9, 10, 11, 12, 13, 14, 15, 16, 17, 18, 19, or 20. In some embodiments, each R⁴ is independently a Cₙ₆ alkylenyl wherein any carbon bonded to two other carbons is optionally independently replaced by N, S, or O, and carbons are optionally independently substituted; in some such embodiments, n3 is 1. In some embodiments, n3 is 1 and R⁴ is C₁-C₅ alkylenyl, optionally methylene. In some embodiments, each R⁴ is independently —C(R^{aa})H—, wherein each R^{aa} is independently the sidechain of a proteinogenic amino acid or the sidechain of an alpha amino acid from Table 1. In some embodiments, each R⁴ is independently a proteinogenic amino acid or an amino acid from Table 1 omitting the backbone amino and carboxylic acid groups of the amino acid.

In some embodiments where n3 is not zero, each L⁴ is independently —N(R^{L4a})—C(O)— or —C(O)—N(R^{L4a})— wherein each R^{L4a} is independently H, methyl, or ethyl. In other such embodiments, each R^{L4a} is independently H or methyl. In other such embodiments, at least one R^{L4a} is ethyl. In some embodiments, each L⁴ is independently —NHC(O)— or —C(O)—NH—. In some embodiments, each L⁴ is —NHC(O)—. In some embodiments, at least one L⁴ is —S—. In some embodiments, at least one L⁴ is —NH—C(O)—NH—. In some embodiments, at least one L⁴ is —NH—C(S)—NH—. In some embodiments, at least one L⁴ is

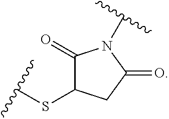 [SMILES: *SC1CC(=O)N(*)C1=O]

In some embodiments, at least one L⁴ is

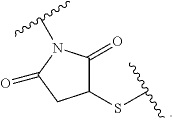 [SMILES: *SC1CC(=O)N(*)C1=O]

In some embodiments, at least one L⁴ is

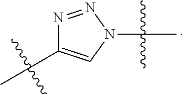 [SMILES: *c1cn(*)nn1]

In some embodiments, at least one L⁴ is

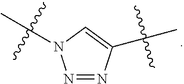 [SMILES: *c1cn(*)nn1]

In some embodiments, n3 is 1, R⁴ is methylene and L⁴ is —NHC(O)—. In some such embodiments, L³ is —NHC(O)—. In some such embodiments, n2 is 1.

In some embodiments where n3 is not zero, each L⁴ is independently —N(R^{L3a})—C(O)— or C(O)—N(R^{L3a})— wherein each R^{L3a} is independently H, methyl, or ethyl. In other such embodiments, each R^{L4a} is independently H or methyl. In other such embodiments, at least one R^{L3a} is ethyl.

X^{br} is a branching atom, i.e. the point where the linker diverges from a single chain to two chains to connect both R^{rad} and R^{alb} to the PSMA-binding moiety. In some embodiments, X^{br} is N, C or CH. In some embodiments, X^{br} is CH. In other embodiments, X^{br} is N.

In some embodiments, X^{br} is separated from ring A by at least 4 ms, at least 5 atoms, at least 6 atoms, at least 7 atoms, at least 8 atoms, at least 9 atoms, or at least 10 atoms. The expression “X^{br} is separated from ring A by at least [number] atoms” refers to the number of atoms that form a contiguous chain by the shortest route between X^{br} and ring A, and excluding X^{br} and ring A atoms from the atom count. The expression “by the shortest route” in this context refers to the possibility for a ring to be included in the atoms separating X^{br} and ring A, such that there are two or more non-equivalent routes to count atoms in a contiguous chain; in such a situation, the shortest route is counted. The number of atoms separating X^{br} and ring A does not include hydrogens and does not include any non-hydrogen atoms branching off the shortest route. For example, in the structure CCZ02017 (see Examples for its chemical structure), the number of atoms separating X^{br} and ring A is 6, and excludes the two amide oxygens and excludes all hydrogens.

In some embodiments, X^{br} is separated from ring A by 6 atoms. In some embodiments, X^{br} is separated from ring A by 5 atoms. In some embodiments, X^{br} is separated from ring A by 4 atoms.

As defined by Formula I, R^{5a}—X^{br}(R^{5c})—R^{5b} forms a Cₙ₇ alkylenyl, alkenylenyl and/or alkynylenyl, wherein n7 is 1-20 (i.e. 1, 2, 3, 4, 5, 6, 7, 8, 9, 10, 11, 12, 13, 14, 15, 16, 17, 18, 19, or 20), wherein any carbon bonded to two other carbons may be independently replaced by N, S, or O heteroatoms. In some embodiments, R^{5a}—X^{br}(R^{5c})—R^{5b} forms a Cₙ₇ alkylenyl. In some embodiments, n7 is 1-15 or 1-10. In some embodiments, 1 carbon is replaced by N, S, or O. In other embodiments, 2, 3, 4, or 5 carbons are each independently replaced by N, S, or O. In some embodiments, the carbons are unsubstituted. In other embodiments, 1 carbon is substituted. In other embodiments, 2 or 3 carbons are independently substituted. The substitutions are as defined in Formula I. In some embodiments, the substitutions are independently selected from one or more than one of hydroxyl, sulfhydryl, amine, guanidino, and/or carboxylic acid. In some embodiments, R^{5a} is absent. In some embodiments, R^{5b} is absent. In some embodiments, R^{5c} is absent. In some embodiments, R^{5a} and R^{5b} are absent. In some embodiments, R^{5a} and R^{5c} are absent. In some embodiments, R^{5b} and R^{5c} are absent. In some embodiments, all three of R^{5a}, R^{5b}, and R^{5c} are absent. In some embodiments, R^{5a} is C₁-C₆ alkylenyl, optionallly —(CH₂)₁₋₄—. In some embodiments, R^{5b} is C₁-C₆ alkylenyl, optionallly —(CH₂)₁₋₄—. In some embodiments, R^{5c} is C₁-C₆ alkylenyl, optionallly —(CH₂)₁₋₄—.

In some embodiments: R^{5a} is absent or C₁-C₆ alkylenyl, optionally —(CH₂)₁₋₄—; R^{5b} is absent or C₁-C₆ alkylenyl, optionally —(CH₂)₁₋₄—; R^{5c} is absent or C₁-C₆ alkylenyl, optionally —(CH₂)₁₋₄—; and X^{br} is N, C or CH; wherein any of R^{5a}, R^{5b} or R^{5c} is absent. In some embodiments, R^{5a}—X^{br}(R^{5c})—R^{5b} forms:

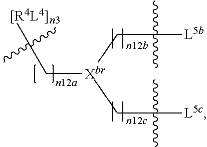

wherein each of n12a, n12b, and n12c is independently 0-4. In some embodiments, R^{5a}—X^{br}(R^{5c})—R^{5b} is

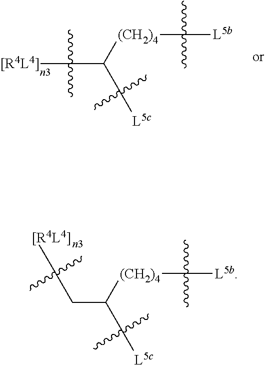

In some embodiments, L^{5b} is —N(R^{L5b})—C(O)— or —C(O)—N(R^{L5b})— wherein R^{L5b} is H, methyl, or ethyl. In some such embodiments, R^{L5b} is H. In other such embodiments, R^{L5b} is methyl. In other such embodiments, R^{L5b} is ethyl. In some embodiments, L^{5b} is —NHC(O)—. In some embodiments, L^{5b} is —C(O)—NH—. In some embodiments, L^{5b} is —S—.

In some embodiments, L^{5b} is —N(R^{L3a})—C(O)— or —C(O)—N(R^{L3a})— wherein R^{L3a} is H, methyl, or ethyl. In some such embodiments, R^{L3a} is H. In other such embodiments, R^{L3a} is methyl. In other such embodiments, R^{L3a} is ethyl. In some embodiments, L^{5b} is —NHC(O)—.

In some embodiments, L^{5b} is —NH—C(O)—NH—. In some embodiments, L^{5b} is —NH—C(S)—NH—. In some embodiments, L^{5b} is

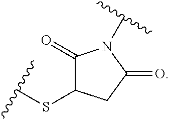 [SMILES: *SC1CC(=O)N(*)C1=O]

In some embodiments, L^{5b} is

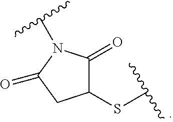 [SMILES: *SC1CC(=O)N(*)C1=O]

In some embodiments, L^{5b} is

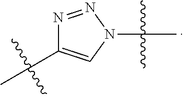 [SMILES: *c1cn(*)nn1]

In some embodiments, L^{5b} is

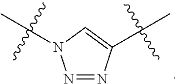 [SMILES: *c1cn(*)nn1]

In some embodiments, L^{5c} is —N(R^{L50})—C(O)— or —C(O)—N(R^{L50})— wherein R^{L5c} is H, methyl, or ethyl. In other such embodiments, R^{L5c} is methyl. In other such embodiments, R^{L5c} is ethyl. In some embodiments, L^{5c} is —NHC(O)—. In some embodiments, L^{5c} is —C(O)—NH—. In some embodiments, L^{5c} is —S—.

In some embodiments, L^{5c} is —N(R^{L3a})—C(O)— or —C(O)—N(R^{L3a})— wherein R^{L3a} is H, methyl, or ethyl. In other such embodiments, R^{L3a} is methyl. In other such embodiments, R^{L3a} is ethyl. In some embodiments, L^{5c} is —NHC(O)—. In some embodiments, L^{5c} is —C(O)—NH—. In some embodiments, L^{5c} is —S—. In some embodiments, L^{5c} is —NH—C(O)—NH—.

In some embodiments, L^{5c} is —NH—C(O)—NH—. In some embodiments, L^{5c} is —NH—C(S)—NH—.

In some embodiments, L^{5c} is

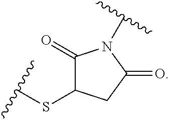 [SMILES: *SC1CC(=O)N(*)C1=O]

In some embodiments, L^{5c} is

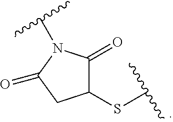 [SMILES: *SC1CC(=O)N(*)C1=O]

some embodiments, L^{5c} is

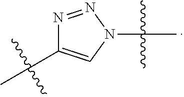 [SMILES: *c1cn(*)nn1]

In some embodiments, L^{5c} is

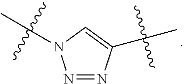 [SMILES: *c1cn(*)nn1]

In some embodiments: n3 is 0; R^{5a} is methylene; X^{br} is N or CH; and R^{5b} is —(CH₂)₄— and R^{5c} is absent, or R^{5c} is —(CH₂)₄— and R^{5b} is absent. In some such embodiments, L^{5b} is —NHC(O)—. In some such embodiments, L^{5c} is —NHC(O)—. L^{5b} and L^{5c} may each be —NHC(O)—.

In some embodiments, n4 is 0. In some embodiments, n4 is 1. In some embodiments n4 is 2. In some embodiments, n4 is 3. In some embodiments n4 is 4.

In embodiments where n4 is not zero, each R⁶ is independently a linear, branched, and/or cyclic Cₙ₈ₐ alkylenyl, alkenylenyl and/or alkynylenyl, wherein each n8a is independently 1-20, wherein any carbon bonded to two other carbons is optionally independently replaced by N, S, or O, and carbons are optionally independently substituted. In some embodiments, each n8a is independently 1-15 or 1-10. In alternative embodiments, each n8a is independently 1, 2, 3, 4, 5, 6, 7, 8, 9, 10, 11, 12, 13, 14, 15, 16, 17, 18, 19, or 20. In some embodiments, each R⁶ is independently a Cₙ₈ₐ alkylenyl wherein any carbon bonded to two other carbons is optionally independently replaced by N, S, or O, and carbons are optionally independently substituted; in some such embodiments, n4 is 1. In some embodiments, n4 is 1 and R⁶ is C₁-C₅ alkylenyl, optionally methylene or —(CH₂)₁₋₄—. In some embodiments, each R⁶ is independently —C(R^{aa})H—, wherein each R^{aa} is independently the sidechain of a proteinogenic amino acid or the sidechain of an alpha amino acid from Table 1. In some embodiments, each R⁶ is independently a proteinogenic amino acid or an amino acid from Table 1 omitting the backbone amino and carboxylic acid groups of the amino acid.

In some embodiments where n4 is not zero, each L⁶ is independently —N(R^{L6a})—C(O)— or C(O)—N(R^{L6a})— wherein each R^{L6a} is independently H, methyl, or ethyl. In other such embodiments, each R^{L6a} is independently H or methyl. In some embodiments where n4 is not zero, each L⁶ is independently —N(R^{L3a})—C(O)— or —C(O)—N(R^{L3a})— wherein each R^{L3a} is independently H, methyl, or ethyl. In other such embodiments, each R^{L3a} is independently H or methyl. In other such embodiments, at least one R^{L6a} is ethyl. In some embodiments, each L⁶ is independently-NHC(O)— or —C(O)—NH—. In some embodiments, each L⁶ is —NHC(O)—. In some embodiments, at least one L⁶ is —S—. In some embodiments, at least one L⁶ is —NH—C(O)—NH—. In some embodiments, L⁶ is —NH—C(S)—NH—. In some embodiments, at least one L⁶ is

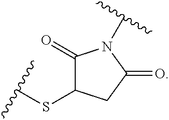 [SMILES: *SC1CC(=O)N(*)C1=O]

In some embodiments, at least one L⁶ is

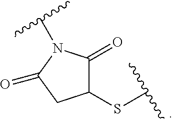 [SMILES: *SC1CC(=O)N(*)C1=O]

In some embodiments, at least one L⁶ is

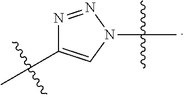 [SMILES: *c1cn(*)nn1]

In some embodiments, at least one L⁶ is

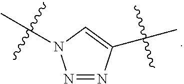 [SMILES: *c1cn(*)nn1]

In some embodiments, n5 is 0. In some embodiments, n5 is 1. In some embodiments n5 is 2. In some embodiments, n5 is 3. In some embodiments n5 is 4.

In embodiments where n5 is not zero, each R⁷ is independently a linear, branched, and/or cyclic C_{n8b} alkylenyl, alkenylenyl and/or alkynylenyl, wherein each n8b is independently 1-20, wherein any carbon bonded to two other carbons is optionally independently replaced by N, S, or O, and carbons are optionally independently substituted. In some embodiments, each n8b is independently 1-15 or 1-10. In alternative embodiments, each n8b is independently 1, 2, 3, 4, 5, 6, 7, 8, 9, 10, 11, 12, 13, 14, 15, 16, 17, 18, 19, or 20. In some embodiments, each R⁷ is independently a C_{n8b} alkylenyl wherein any carbon bonded to two other carbons is optionally independently replaced by N, S, or O, and carbons are optionally independently substituted; in some such embodiments, n5 is 1. In some embodiments, n5 is 1 and R⁷ is C₁-C₅ alkylenyl, optionally methylene or —(CH₂)₁₋₄—. In some embodiments, each R⁷ is independently —C(R^{aa})H—, wherein each R^{aa} is independently the sidechain of a proteinogenic amino acid or the sidechain of an alpha amino acid from Table 1. In some embodiments, each R⁶ is independently a proteinogenic amino acid or an amino acid from Table 1 omitting the backbone amino and carboxylic acid groups of the amino acid.

In some embodiments where n5 is not zero, each L⁷ is independently —N(R^{L7a})—C(O)— or C(O)—N(R^{L7a})— wherein each R^{L7a} is independently H, methyl, or ethyl. In other such embodiments, each R^{L7a} is independently H or methyl. In other such embodiments, at least one R^{L7a} is ethyl. In some embodiments, each L⁷ is independently —NHC(O)— or —C(O)—NH—. In some embodiments, each L⁷ is —NHC(O)—. In some embodiments where n5 is not zero, each L⁷ is independently —N(R^{L3a})—C(O)— or —C(O)—N(R^{L3a})— wherein each R^{L3a} is independently H, methyl, or ethyl. In other such embodiments, each R^{L3a} is independently H or methyl. In other such embodiments, at least one R^{L3a} is ethyl. In some embodiments, each L⁷ is independently —NHC(O)— or —C(O)—NH—. In some embodiments, each L⁷ is —NHC(O)—. In some embodiments, at least one L⁷ is —S—. In some embodiments, L⁷ is —NH—C(O)—NH.

In some embodiments, L⁷ is —NH—C(S)—NH—. In some embodiments, at least one L⁷ is

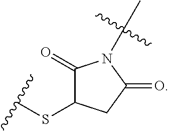 [SMILES: *SC1CC(=O)N(*)C1=O]

In some embodiments, at least one L⁷ is

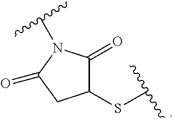 [SMILES: *SC1CC(=O)N(*)C1=O]

In some embodiments, at least one L⁷ is

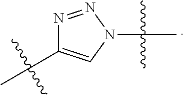 [SMILES: *c1cn(*)nn1]

In some embodiments, at least one L⁷ is

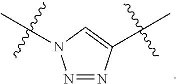 [SMILES: *c1cn(*)nn1]

R^{rad} is a radiometal chelator that is separated from ring A by at least 7 atoms. The expression “R^{rad} is separated from ring A by at least [number] atoms” refers to the number of atoms that form a contiguous chain by the shortest route between R^{rad} and ring A, and excluding R^{rad} and ring A atoms from the atom count. The expression “by the shortest route” in this context refers to the possibility for a ring to be included in the atoms separating R^{rad} and ring A, such that there are two or more non-equivalent routes to count atoms in a contiguous chain; in such a situation, the shortest route is counted. The number of atoms separating R^{rad} and ring A does not include hydrogens and does not include any non-hydrogen atoms branching off the shortest route. For example, in the structure CCZ02017 (see Examples for chemical structure), the number of atoms separating R^{rad} and ring A is 13 (including the linking amide attached to DOTA), and excludes the three amide oxygens, excludes the branch of the linker connecting R^{alb}, and excludes all hydrogens. In some embodiments, R^{rad} is separated from ring A by at least 7 atoms, at least 8 atoms, at least 9 atoms, at least 10 atoms, at least 11 atoms, at least 12 atoms, at least 13 atoms, at least 14 atoms, or at least 15 atoms. In some embodiments, R^{rad} is separated from ring A by 7-18 atoms (e.g. 7, 8, 9, 10, 11, 12, 13, 14, 15, 16, 17, or 18 atoms). In some embodiments, R^{rad} is bound by a radiometal. In some embodiments, the radiometal is not bound to R^{rad}.

The various embodiments described herein can also be combined to form more specific embodiments as needed. For example, in a specific embodiment, ring A is

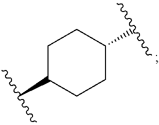

### R² is

—(CH₂)₄—; R³ is:

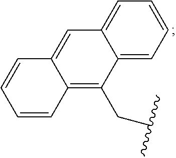 [SMILES: *Cc1c2ccccc2cc2ccccc12]

n1 is 0; L¹ is —N(R^{L1a})—C(O)—, or —C(O)—N(R^{L1a})—, wherein R^{L1a} is H; L² is —N(R^{L2a})—C(O)—, or —C(O)—N(R^{L2a})—, wherein R^{L2a} is H; and R¹ is —R^{1a}R^{1b}—, wherein R^{1a} is —CH₂—, and R^{1b} is —CH₂— or —CHF—.

In another specific embodiment, R^{5c} is absent; L^{5c} is —N(R^{L3a})—C(O)— or —C(O)—N(R^{L3a})—, wherein R^{L3a} is H; R⁷ is methylene; L⁷ is —N(R^{L3a})—C(O)— or —C(O)—N(R^{L3a})—, wherein R^{L3a} is H;
n5 is 1; and R^{alb} is:

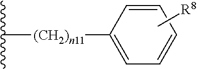

wherein n11 is 3 and R⁸ is OCH₃, or NO₂.

In another specific embodiment, n2 is 0 or 1; L³ is —N(R^{L3a})—C(O)—, or —C(O)—N(R^{L3a})—, wherein R^{L3a} is H; R⁴ is methylene; L⁴ is —N(R^{L3a})—C(O)—, or —C(O)—N(R^{L3a})—, wherein R^{L3a} is H; n3 is 1; R^{5a} is absent; and X^{br} is CH.

In another specific embodiment, R^{5b} is a linear C₁-C₆ alkylenyl; L^{5b} is —N(R^{L3a})—, C(O)— or —C(O)—N(R^{L3a})—, wherein R^{L3a} is H; R⁶ is methylene; L⁶ is —N(R^{L3a})—C(O)— or —O(O)—N(R^{L3a})—, wherein R^{L3a} is H; and n4 is 0-2.

In some embodiments, R^{rad} is selected from Table 2, wherein R^{rad} is optionally bound by a radiometal.

The radiometal chelator may be any radiometal chelator suitable for binding to the radiometal and which is functionalized for attachment to an amino group. Many suitable radiometal chelators are known, e.g. as summarized in Price and Orvig, Chem. Soc. Rev., 2014, 43, 260-290, which is incorporated by reference in its entirety. Non-limiting examples of radiometal chelators include chelators selected from the group consisting of: DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; and H₆phospa. Exemplary non-limiting examples of radiometal chelators and example radioisotopes chelated by these chelators are shown in Table 2. In Table 2, the functional groups for linkage are shown in their non-linked forms; the person of skill in the art would appreciate that once linked to the compounds disclosed herein, these linking functional groups would be modified (e.g. COOH or NH₂ in the chelator would become an amide linkage when reacted with NH₂ or COOH, respectively, in the linker). When counting the atoms separating R^{rad} from ring A, the linkage atoms (or linkage-forming functional groups in Table 2) are not included in the atom count. In alternative embodiments, R^{rad} comprises a radiometal chelator selected from those listed above or in Table 2 linked via a linkage-forming functional group (e.g. COOH, NH₂, SH, and the like). One skilled in the art could replace any of the chelators listed herein with another chelator.

TABLE 2
Exemplary chelators and exemplary isotopes which bind said chelators.

| Chelator | Isotopes |
|---|---|
| 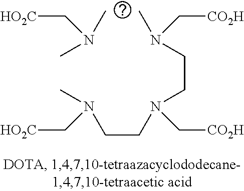 | Cu-64/67 In-111 Lu-177 Y-86/90 Bi-203/212/213 Pb-212 Ac-225 Gd-159 Yb-175 Ho-166 As-211 Sc-44/47 |
| | Pm-149 |
| | Pr-142 |
| | Sn-117m |
| | Sm-153 |
| | Tb-149/161 |
| | Er-165 |
| | Ra-223/224 |
| | Th-227 |
| 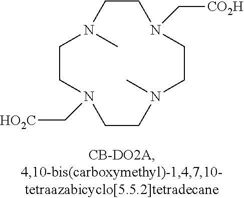 [SMILES: CN1CCN(CC(=O)O)CCN(C)CCN(CC(=O)O)CC1] | Cu-64/67 |
| 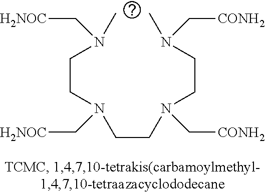 [SMILES: CN(CCN(CCN(CCN(C)CC(N)=O)CC(N)=O)CC(N)=O)CC(N)=O] | Pb-212 |
| 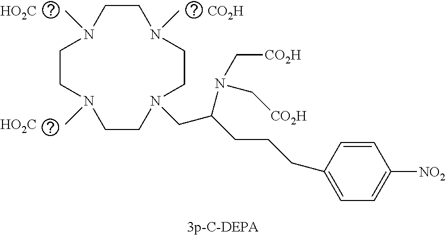 | Bi-212/213 |
| 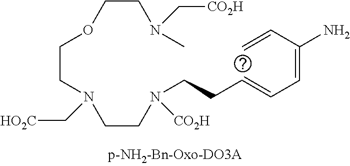 | Cu-64/67 |
| 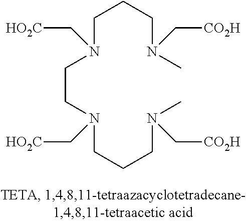 [SMILES: CN(CCCN(CCN(CCCN(C)CC(=O)O)CC(=O)O)CC(=O)O)CC(=O)O] | Cu-64/67 |
| 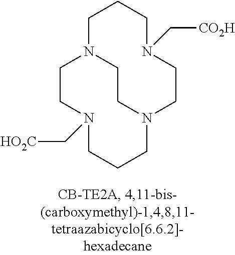 [SMILES: O=C(O)CN1CCCN2CCN(CCCN(CC(=O)O)CC2)CC1] | Cu-64/67 |
| 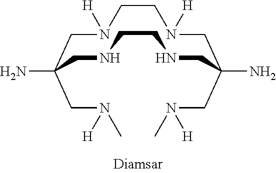 | Cu-64/67 |
| 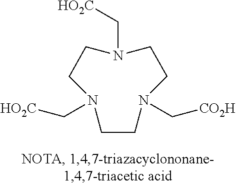 [SMILES: O=C(O)CN1CCN(CC(=O)O)CCN(CC(=O)O)CC1] | Cu-64/67 In-111 Sc-44/47 |
| 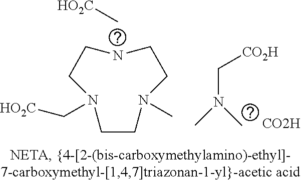 | Cu-64/67 Lu-177 Y-86/90 Bi-213 Pb-212 |
| 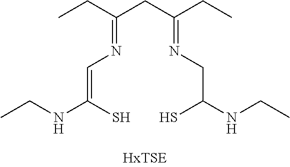 [SMILES: CCN/C(S)=C/N=C(\CC)C/C(CC)=N/CC(S)NCC] | Au-198/199 |
| 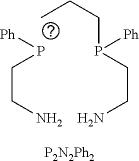 | Rh-105 |
| 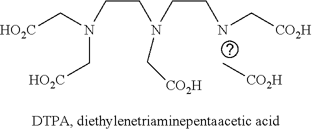 | In-111 Sc-44/47 Lu-177 Y-86/90 Sn-117m Pd-109 |
| 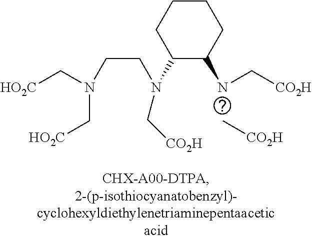 | In-111 Lu-177 Y-86/90 Bi-212/213 |
| 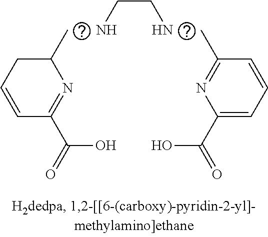 | Cu-64/67 |
| 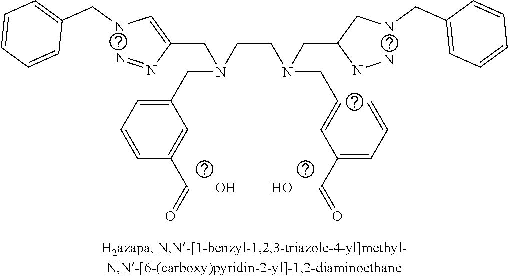 | Cu-64/67 |
| 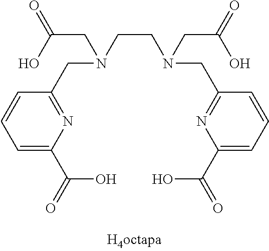 [SMILES: O=C(O)CN(CCN(CC(=O)O)Cc1cccc(C(=O)O)n1)Cc1cccc(C(=O)O)n1] | In-111 Lu-177 Y-86/90 Ac-225 |
| 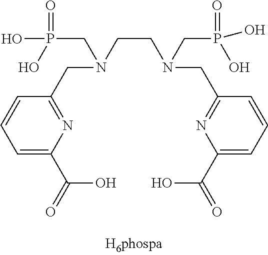 [SMILES: O=C(O)c1cccc(CN(CCN(Cc2cccc(C(=O)O)n2)CP(=O)(O)O)CP(=O)(O)O)n1] | Ac-225 |
| 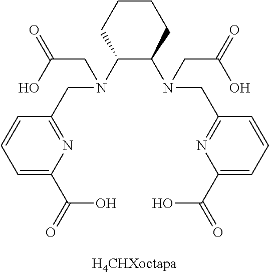 [SMILES: O=C(O)CN(Cc1cccc(C(=O)O)n1)[C@@H]1CCCC[C@H]1N(CC(=O)O)Cc1cccc(C(=O)O)n1] | In-111 Ac-225 |
| 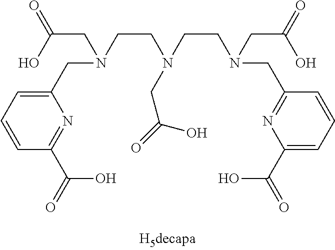 [SMILES: O=C(O)CN(CCN(CC(=O)O)Cc1cccc(C(=O)O)n1)CCN(CC(=O)O)Cc1cccc(C(=O)O)n1] | In-111 Lu-177 Ac-225 |
| 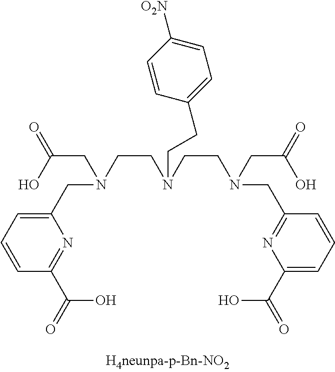 [SMILES: O=C(O)CN(CCN(CCc1ccc([N+](=O)[O-])cc1)CCN(CC(=O)O)Cc1cccc(C(=O)O)n1)Cc1cccc(C(=O)O)n1] | In-111 Lu-177 Ac-225 |
| 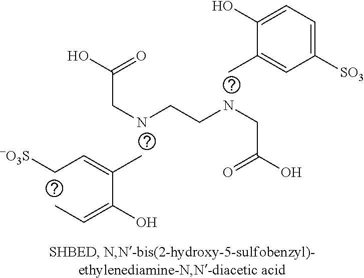 | In-111 |
| 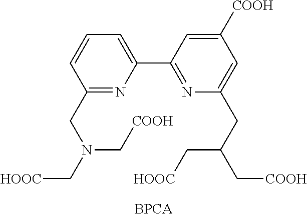 [SMILES: O=C(O)CC(CC(=O)O)Cc1cc(C(=O)O)cc(-c2cccc(CN(CC(=O)O)CC(=O)O)n2)n1] | In-111 |
| 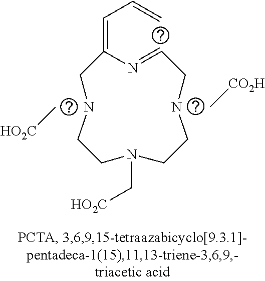 | Cu-64/67 |
| 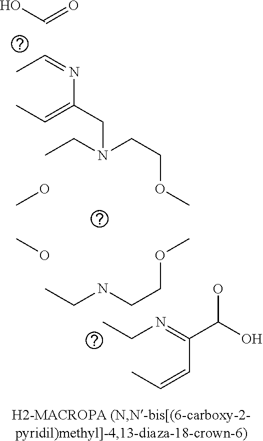 | Ac-225 |
| 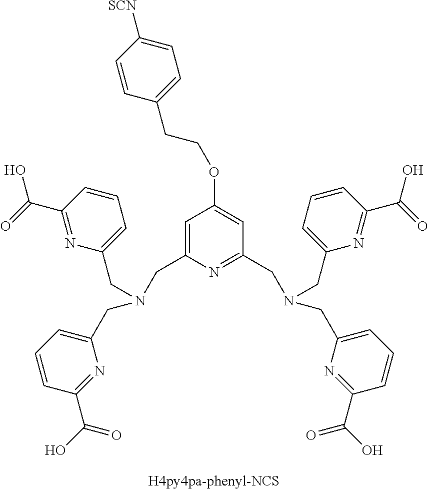 | Ac-225 Tb-161 |
| 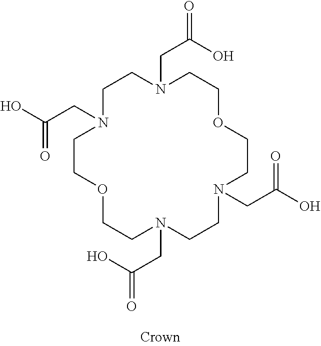 [SMILES: O=C(O)CN1CCOCCN(CC(=O)O)CCN(CC(=O)O)CCOCCN(CC(=O)O)CC1] | Ac-225 Tb-161 Lu-177 |
| 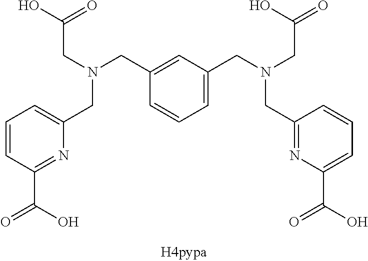 [SMILES: O=C(O)CN(Cc1cccc(CN(CC(=O)O)Cc2cccc(C(=O)O)n2)c1)Cc1cccc(C(=O)O)n1] | Lu-177 |

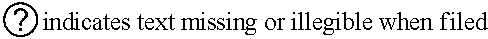

In some embodiments, the chelator is DOTA, or a derivative thereof, linked via an amide (e.g. formed from one of the carboxyl groups shown in Table 2. In some embodiments, the chelator is CB-DO2A, or a derivative thereof, linked via an amide (e.g. formed from one of the carboxyl groups shown in Table 2). In some embodiments, the chelator is TCMC, or a derivative thereof, linked via an amide (e.g. formed from one of the —CONH₂ groups shown in Table 2). In some embodiments, the chelator is 3p-C-DEPA, or a derivative thereof, linked via an amide (e.g. formed from one of the carboxyl groups shown in Table 2). In some embodiments, the chelator is p-NH₂-Bn-Oxo-DO3A or a derivative thereof, linked via an amide (e.g. formed from one of the carboxyl groups shown in Table 2). In some embodiments, the chelator is TETA, or a derivative thereof, linked via an amide (e.g. formed from one of the carboxyl groups shown in Table 2). In some embodiments, the chelator is CB-TE2A, or a derivative thereof, linked via an amide (e.g. formed from one of the carboxyl groups shown in Table 2). In some embodiments, the chelator is Diamsar, or a derivative thereof, linked via an amide (e.g. formed from one of the amino groups shown in Table 2), In some embodiments, the chelator is NOTA, or a derivative thereof, linked via an amide (e.g. formed from one of the carboxyl groups shown in Table 2). In some embodiments, the chelator is NETA, or a derivative thereof, linked via an amide (e.g. formed from one of the carboxyl groups shown in Table 2). In some embodiments, the chelator is HxTSE, or a derivative thereof, linked via an amide (e.g. formed from one of the amino groups shown in Table 2). In some embodiments, the chelator is P₂N₂Ph₂, or a derivative thereof, linked via an amide (e.g. formed from one of the amino groups shown in Table 2). In some embodiments, the chelator is DTPA, or a derivative thereof, linked via an amide (e.g. formed from one of the carboxyl groups shown in Table 2). In some embodiments, the chelator is CHX-A00-DTPA, or a derivative thereof, linked via an amide (e.g. formed from one of the carboxyl groups shown in Table 2). In some embodiments, the chelator is H₂dedpa, or a derivative thereof, linked via an amide (e.g. formed from one of the carboxyl groups shown in Table 2). In some embodiments, the chelator is H₂azapa, or a derivative thereof, linked via an amdie (e.g. formed from one of the carboxyl groups shown in Table 2). In some embodiments, the chelator is H₄octapa, or a derivative thereof, linked via an amide (e.g. formed from one of the carboxyl groups shown in Table 2). In some embodiments, the chelator is H₆phospa, or a derivative thereof, linked via an amide (e.g. formed from one of the carboxyl groups shown in Table 2). In some embodiments, the chelator is H₄CHXoctapa, or a derivative thereof, linked via an amide (e.g. formed from one of the carboxyl groups shown in Table 2). In some embodiments, the chelator is H₅decapa, or a derivative thereof, linked via an amide (e.g. formed from one of the carboxyl groups shown in Table 2). In some embodiments, the chelator is H₄neunpa-p-Bn-NO2, or a derivative thereof, linked via an amide (e.g. formed from one of the carboxyl groups shown in Table 2). In some embodiments, the chelator is SHBED, or a derivative thereof, linked via an amide (e.g. formed from one of the carboxyl groups shown in Table 2). In some embodiments, the chelator is BPCA, or a derivative thereof, linked via an amide (e.g. formed from one of the carboxyl groups shown in Table 2). In some embodiments, the chelator is PCTA, or a derivative thereof, linked via an amide (e.g. formed from one of the carboxyl groups shown in Table 2). In some embodiments, the chelator is H₂-MACROPA, or a derivative thereof, linked via an amide (e.g. formed from one of the carboxyl groups shown in Table 2). In some embodiments, the chelator is Crown, or a derivative thereof, linked via an amide (e.g. formed from one of the carboxyl groups shown in Table 2).

In some embodiments, the radiometal chelator is a derivative of a radiometal chelator shown in Table 2. A derivative may include, e.g. (1) modification of a functional group of the chelator (e.g. a carboxyl group, an amino group, etc.) or (2) attachment of a new functional group (e.g. attachment of an R-group to an ethylene carbon located between two nitrogen atoms, wherein the R-group is a functional group fused to a spacer). In some embodiments, a carboxyl functional group shown in Table 2 is replaced with azidopropyl ethylacetamide (e.g. azido-mono-amide-DOTA), butynylacetamide (e.g. butyne-DOTA), thioethylacetamide (e.g. DO3A-thiol), maleimidoethylacetamide (e.g. maleimido-mono-amide-DOTA), or N-hydroxysuccinimide ester (e.g. DOTA-NHS-ester). When linked, these derivative chelators can be linked either via an amide (formed from a remaining carboxyl group) or via —C(O)—NH—(CH₂)₂₋₃-(triazole) or —C(O)—NH—(CH₂)₂₋₃-(thiomaleimide). In other embodiments, a backbone carbon (e.g. in an ethylene positioned between two backbone nitrogen atoms) in the chelator ring is fused to an R-group containing a functional group, optionally wherein the R-group is —(CH₂)₁₋₃-(phenyl)-N═C═S or —(CH₂)₁₋₃-(phenyl)-N═C═O, optionally 1,4-isothiocyanatobenzyl; e.g. p-SCN-Bn-DOTA (S—2-(4-isothiocyanatobenzyl)-1,4,7,10-tetraazacyclododecane tetraacetic acid), p-SCN-Bn-NOTA (2-S-(4-isothiocyanatobenzyl)-1,4,7-triazacyclononane-1,4,7-triacetic acid), and the like. When linked, these derivatives can form a urea linkage (formed from isocyanate) or a thiourea linkage (formed from isothiocyanate).

In some embodiments, the radiometal chelator is conjugated with a radioisotope (i.e. radiometal). The conjugated radioisotope may be, without limitation, ¹⁶⁵Er, ²¹²Bi, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb ¹⁴²Pr, ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ²²⁴Ra, ²²⁷Th, ²²³Ra, ⁶⁴Cu, ⁶⁷Cu, and the like. In some embodiments, the chelator is a chelator from Table 2 and the conjugated radioisotope is a radioisotope indicated in Table 2 as a binder of the particular chelator.

In some embodiments, the chelator is: DOTA or a derivative thereof, optionally conjugated with ¹⁷⁷Lu, ¹¹¹In ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ¹⁶⁵Er, ²²⁴Ra, ²¹²Bi, ²²⁷Th, ²²³Ra, ⁶⁴Cu or ⁶⁷Cu; Crown optionally conjugated with ²²⁵Ac, ²²⁷Th or ¹⁷⁷Lu; ¹⁵³Sm, MACROPA optionally conjugated with ²²⁵Ac; Me-3,2-HOPO optionally conjugated with ²²⁷Th; H₄py4pa optionally conjugated with ²²⁵Ac; H₄pypa optionally conjugated with ¹⁷⁷Lu; or DTPA optionally conjugated with ¹¹¹In.

In some embodiments, the chelator is TETA (1,4,8,11-tetraazacyclotetradecane-1,4,8,11-tetraacetic acid), SarAr (1-N-(4-Aminobenzyl)-3, 6,10,13,16,19-hexaazabicyclo[6.6.6]-eicosane-1,8-diamine), NOTA (1,4,7-triazacyclononane-1,4,7-triacetic acid), TRAP (1,4,7-triazacyclononane-1,4,7-tris[methyl(2-carboxyethyl)phosphinic acid), HBED (N,N′-bis(2-hydroxybenzyl)-ethylenediamine-N,N′-diacetic acid), 2,3-HOPO (3-hydroxypyridin-2-one), PCTA (3,6,9,15-tetraazabicyclo[9.3.1]-pentadeca-1 (15),11,13-triene-3,6,9,-triacetic acid), DFO (desferrioxamine), DTPA (diethylenetriaminepentaacetic acid), OCTAPA (N,N′-bis(6-carboxy-2-pyridylmethyl)-ethylenediamine-N,N′-diacetic acid) or another picolinic acid derivative.

In some embodiments, the radiometal chelator is mercaptoacetyl, hydrazinonicotinamide, dimercaptosuccinic acid, 1,2-ethylenediylbis-L-cysteine diethyl ester, methylenediphosphonate, hexamethylpropyleneamineoxime, or hexakis(methoxy isobutyl isonitrile). In some of these embodiments, the chelator is bound by a radioisotope. In some such embodiments, the radioisotope is ¹⁸⁶Re or ¹⁸⁸Re. In some embodiments, the chelator is not bound by a radioisotope.

R^{alb} is an albumin binder. In some embodiments, R^{alb} is —(CH₂)ⁿ⁹—CH₃ wherein n9 is 8-20; in alternative embodiments, n9 is 8, 9, 10, 11, 12, 13, 14, 15, 16, 17, 18, 19, or 20. In some embodiments, R^{a1b} is —(CH₂)ⁿ¹⁰—C(O)OH wherein n10 is 8-20; in alternative embodiments, n10 is 8, 9, 10, 11, 12, 13, 14, 15, 16, 17, 18, 19, or 20. In some embodiments, R^{alb} is

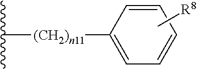

wherein n11 is 1-4 and R⁸ is I, Br, F, Cl, H, OH, OCH₃, NH₂, NO₂, or CH₃; in alternative embodiments, n11 is 1, 2, 3, or 4. In some embodiments, n11 is 3. In some embodiments, R⁸ is OCH₃ or NO₂. In some embodiments, R^{alb} is

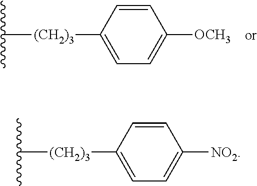

R^{alb} is separated from ring A by at least 7 atoms. In some embodiments, R^{alb} is separated from ring A by at least 4 atoms, at least 5 atoms, at least 6 atoms, at least 7 atoms, at least 8 atoms, at least 9 atoms, or at least 10 atoms. The expression “R^{alb} is separated from ring A by at least [number] atoms” refers to the number of atoms that form a contiguous chain by the shortest route between R^{alb} and ring A, and excluding R^{alb} and ring A atoms from the atom count. The expression “by the shortest route” in this context refers to the possibility for a ring to be included in the atoms separating R^{alb} and ring A, such that there are two or more non-equivalent routes to count atoms in a contiguous chain; in such a situation, the shortest route is counted. The number of atoms separating R^{alb} and ring A does not include hydrogens and does not include any non-hydrogen atoms branching off the shortest route. For example, in the structure CCZ02017 (see Examples for chemical structure), the number of atoms separating R^{alb} and ring A is 12, and excludes the four amide oxygens, excludes the branch of the linker that links R^{rad}, and excludes all hydrogens.

In some embodiments, R^{alb} is separated from ring A by 7-18 atoms, and R^{rad} is separated from ring A by 7-18 atoms. In some embodiments, R^{alb} and R^{rad} are each separated from ring A by 10-11 atoms. In some such embodiments, X^{br} is separated from ring A by 4-6, optionally 6 atoms.

In some embodiments, the compound (of Formula I) has Formula II or is a salt or a solvate of Formula II:

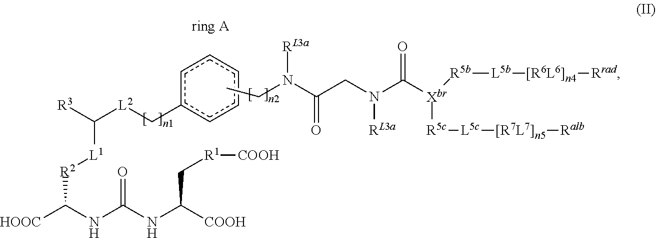

wherein R¹, R², R³, R^{L3a}, R^{5b}, R^{5c}, R⁶, R⁷, R^{rad}, R^{alb}, L¹, L², L^{5b}, L^{5c}, L⁶, L⁷, ring A, n1, n2, n4, and n5 are as defined in Formula I, or as defined in any other embodiment(s) defined herein. In some of these embodiments: R¹ is —CH₂—CH₂— or —CHF—. In some of these embodiments, R² is —(CH₂)₄—. In some of these embodiments, L¹ is —NH—C(O)—. In some of these embodiments, R³ is

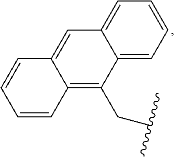 [SMILES: *Cc1c2ccccc2cc2ccccc12]

optionally wherein R³ is

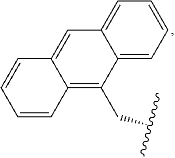

In some of these embodiments, L² is —NHC(O)—. In some of these embodiments, n1 is 0; ring A has 0 double bonds and is bonded at para position, optionally wherein ring A is

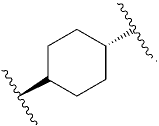

In some of these embodiments, n2 is 0 or 1. In some of these embodiments, each R^{L3a} is H. In some of these embodiments, X^{br} is N, C, or CH. In some of these embodiments: R^{5b} is —(CH₂)₄— and R^{5c} is absent; or R^{5c} is —(CH₂)₄— and R^{5b} is absent. In some of these embodiments, L^{5b} is —NH—C(O)—. In some of these embodiments, L^{5c} is —NH—C(O)—. In some of these embodiments, L^{5b} is —NH—C(O)—. In some of these embodiments, n4 is 0 or 1, and R⁶—when present—is methylene. In some of these embodiments, n5 is 0 or 1, and R⁷—when present—is methylene. In some of these embodiments, L⁶-when present—is —NH—C(O)—. In some of these embodiments, L⁷-when present—is —NH—C(O)—. In some of these embodiments, R^{alb} is

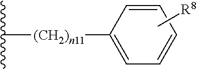

wherein n11 is 3 and R⁸ is OCH₃ or NO₂. In some of these embodiments, R^{rad} is DOTA or a DOTA derivative.

In some embodiments, the compound is CCZ02009, CCZ02017, CCZ02008, CCZ02025, CCZ02024, CCZ02015, CCZ02019, CCZ02012, or CCZ02013, (see Examples for chemical structures) or a salt or solvate thereof, optionally conjugated with ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ¹⁶⁵Er, ²²⁴Ra, ²¹²Bi, ²²⁷Th, ²²³Ra, ⁶⁴Cu or ⁶⁷Cu.

In some embodiments, the compound is CCZ02009, CCZ02017, CCZ02008, CCZ02025, CCZ02024, CCZ02015, CCZ02019, CCZ02012, CCZ02005, CCZ02021, CCZ02022, CCZ02059, CCZ02060, CCZ02034, CCZ02061, or CCZ02013, (see Examples for chemical structures) or a salt or solvate thereof, optionally conjugated with ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ¹⁶⁵Er, ²²⁴Ra, ²¹²Bi, ²²⁷Th, ²²³Ra, ⁶⁴Cu or ⁶⁷Cu.

In some embodiments, the compound is CCZ02005, CCZ02021, CCZ02022, CCZ02059, CCZ02060, CCZ02034, CCZ02061, or a salt or solvate thereof, optionally conjugated with ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ¹⁶⁵Er, ²²⁴Ra, ²¹²Bi, ²²⁷Th, ²²³Ra, ⁶⁴Cu or ⁶⁷Cu.

When the radiometal chelator is conjugated with a therapeutic radioisotope (e.g. ¹⁶⁵Er, ²¹²Bi, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ²²⁴Ra, ²²⁷Th, ²²³Ra, ⁶⁴Cu, ⁶⁷Cu, or the like), there is disclosed the use of certain embodiments of the compound (or a pharmaceutical composition thereof) for the treatment of PSMA-expressing conditions or diseases (e.g. tumors and the like) in a subject.

Accordingly, there is provided use of the compound in preparation of a medicament for treating a PSMA-expressing condition or disease in a subject. There is also provided a method of treating PSMA-expressing disease in a subject, in which the method comprises: administering to the subject a composition comprising the compound and a pharmaceutically acceptable excipient. For example, but without limitation, the disease may be a PSMA-expressing tumor or a PSMA-expressing cancer.

PSMA expression has been detected in various cancers (e.g. Rowe et al., 2015, Annals of Nuclear Medicine 29:877-882; Sathekge et al., 2015, Eur J Nucl Med Mol Imaging 42:1482-1483; Verburg et al., 2015, Eur J Nucl Med Mol Imaging 42:1622-1623; and Pyka et al., J Nucl Med Nov. 19, 2015 jnumed.115.164442). Accordingly, without limitation, the PSMA-expressing cancer may be prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma. In some embodiments, the cancer is prostate cancer.

The compounds presented herein incorporate peptides, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g. under mild basic conditions, such as piperidine (20-50% v/v) in DMF. The amino acid to be added must also have been activated for coupling (e.g. at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like. For long peptides or if desired, peptide synthesis and ligation may be used.

Apart from forming typical peptide bonds to elongate a peptide, peptides may be elongated in a branched fashion by attaching to side chain functional groups (e.g. carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF. Deprotected side chains of Asp/Glu (L- or D-forms) and Lys/Orn/Dab/Dap (L- or D-forms) can then be coupled, e.g. by using the coupling reaction conditions described above.

Peptide backbone amides may be N-methylated (i.e. alpha amino methylated) or N-alkylated. This may be achieved by directly using Fmoc-N-methylated amino acids (or Fmoc-N-alkylated amino acids) during peptide synthesis. Alternatively, N-methylation (or N-alkylation) under Mitsunobu conditions may be performed. First, a free primary amine group is protected using a solution of 4-nitrobenzenesulfonyl chloride (Ns-Cl) and 2,4,6-trimethylpyridine (collidine) in NMP. N-methylation may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated alpha amino groups, HATU, HOAt and DIEA may be used.

The PSMA-binding moiety (e.g. Lys-ureido-Glu, Lys-ureido-Aad, and the like) may be constructed on solid phase via the formation of a ureido linkage between the amino groups of two amino acids. This can be done by attaching an Fmoc-protecting amino acid (for example Fmoc-Lys(ivDde)-OH) to Wang resin using standard activation/coupling strategy (for example, Fmoc-protected amino acid (4 eq.), HATU (4 eq.) and N,N-diisopropylethylamine (7 eq.) in N,N-dimethylformamide). The Fmoc-protecting group is then removed by 20% piperidine in N,N-dimethylformamide. To form the ureido linkage, the freed amino group of the solid-phase-attached amino acid is reacted with the 2^{nd} amino acid which has its carboxylate group protected with a t-butyl group and its amino group activated and converted to an isocyanate group (—N═C═O). The activation and conversion of an amino group to an isocyanate group can be achieved by reacting the amino group with phosgene or triphosgene. After the formation of the ureido linkage, the side chain functional group of the amino acid (for example ivDde on Lys) can be removed, and then the linker, albumin-binding motif, and/or radiolabeling group (e.g. radiometal chelator and the like) can be subsequently coupled to the PSMA-binding moiety.

The formation of the thioether (—S—) linkages (e.g. for L¹, L², L³, and the like) can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino)propyl bromide and the like) in an appropriate solvent (such as N,N-dimethylformamide and the like) in the presence of base (such as N,N-diisopropylethylamine and the like). If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

The formation of the linkage (e.g. for L¹, L², L³, and the like) between a thiol group and a maleimide group can be performed using the conditions described above for the formation of the thioether (—S—) linkage simply by replacing the alkyl halide with a maleimide-containing compounds. Similarly, this reaction can be conducted in solid phase or solution phase. If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

Urea or thiourea linkages can be made from reaction of an amine group with an isocyanate or an isothiocyanate, respectively, which are common functional groups on radiometal chelators. The isothiocyanate functional group may be added to the radiometal chelator by reacting an amino group on the chelator with thiophosgene [i.e. C(S)Cl₂]. Similarly, the isocyanate functional group may be added to the radiometal chelator by reacting an amino group on the chelator with phosgene [i.e. C(O)Cl₂].

Non-peptide moieties (e.g. radiometal chelator groups, albumin-binding groups and/or linkers) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moiety may be clicked to the azide-containing peptide in the presence of Cu²⁺ and sodium ascorbate in water and organic solvent, such as acetonitrile (ACN) and DMF and the like. Non-peptide moieties may also be added in solution phase, which is routinely performed.

The synthesis of radiometal chelators is well-known and many chelators are commercially available (e.g. from Sigma-Aldrich™/Milipore Sigma™ and others). Protocols for conjugation of radiometals to the chelators are also well known (e.g. see Examples, below).

When the peptide has been fully synthesized on the solid support, the desired peptide may be cleaved from the solid support using suitable reagents, such as TFA, triisopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e. deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches.

The present invention will be further illustrated in the following examples.

### EXAMPLES

### General Methods

All chemicals and solvents were obtained from commercial sources, and used without further purification. PSMA-targeted peptides were synthesized using solid phase approach on an AAPPTec (Louisville, KY) Endeavor 90 peptide synthesizer. Purification and quality control of cold and radiolabeled peptides were performed on Agilent HPLC systems equipped with a model 1200 quaternary pump, a model 1200 UV absorbance detector (set at 220 nm), and a Bioscan (Washington, DC) NaI scintillation detector. The operation of Agilent HPLC systems was controlled using the Agilent ChemStation software. The HPLC columns used were a semi-preparative column (Luna C18, 5μ, 250×10 mm) and an analytical column (Luna C18, 5μ, 250×4.6 mm) purchased from Phenomenex (Torrance, CA). The HPLC solvents were A: H₂O containing 0.1% TFA, and B: CH₃CN containing 0.1% TFA. The collected HPLC eluates containing the desired peptide were lyophilized using a Labconco (Kansas City, MO) FreeZone 4.5 Plus freeze-drier. Mass analyses were performed using a Waters (Milford, Massachusetts) LC-MS with a QDa mass detector an ESI ion source. C18 Sep-Pak cartridges (1 cm³, 50 mg) were obtained from Waters (Milford, MA). ⁶⁸Ga was eluted from an iThemba Labs (Somerset West, South Africa) generator, and was purified using a DGA resin column from Eichrom Technologies LLC (Lisle, IL). Radioactivity of ⁶⁸Ga or ¹⁷⁷Lu-labeled peptides was measured using a Capintec (Ramsey, NJ) CRC©-25R/W dose calibrator, and the radioactivity of mouse tissues collected from biodistribution studies were counted using a Perkin Elmer (Waltham, MA) Wizard2 2480 automatic gamma counter.

### General Synthesis of PSMA-Targeting Molecules

Peptidomimetic PSMA-targeting Lys-ureido-Glu moiety was synthesized by solid-phase peptide chemistry. Fmoc-Lys(ivDde)-Wang resin was swelled in CH₂Cl₂, followed by Fmoc removal by treating the resin with 20% piperidine in DMF. To generate the isocyanate of the H-Glu(OtBu)-OtBu moiety, a solution of H-Glu(OtBu)-OtBu and diisopropylethylamine in CH₂Cl₂ was cooled to −78° C. in a dry ice/acetone bath. Triphosgene was dissolved in CH₂Cl₂, and the resulting solution was added dropwise to the reaction at −78° C. The reaction was then allowed to warm to room temperature and stirred for 30 minutes. After which the isocyanate of the H-Glu(OtBu)-OtBu solution was added to the lysine-immobilized resin and reacted for 16 h. After washing the resin with DMF, the ivDde-protecting group was removed with 2% hydrazine in DMF. Fmoc-protected amino acids were then coupled to the side chain of Lys in presence of HATU and N,N-diisopropylethylamine. Finally, DOTA-tris(t-bu)ester (2-(4,7,10-tris(2-(t-butoxy)-2-oxoehtyl)-1,4,7,10)-tetraazacyclododecan-1-yl)acetic acid) was coupled. The peptide was then deprotected and simultaneously cleaved from the resin by treating with 95/5 trifluoroacetic acid (TFA)/triisopropylsilane (TIS) for 3-4 h at room temperature. After filtration, the peptide was precipitated by the addition of the TFA solution to cold diethyl ether. The crude peptide was purified by HPLC using the preparative column. The eluates containing the desired peptide were collected, pooled, and lyophilized.

### General Synthesis of Ga or Lu-Labeled Standards

To prepare Ga or Lu-labeled standards, a solution of each precursor was incubated with GaCl₃ or LuCl₃ (5 eq.) in NaOAc buffer (0.1 M, 500 μL, pH 4.2) at 80-90° C. for 15 min. The reaction mixture was then purified by HPLC using the semi-preparative column, and the HPLC eluates containing the desired peptide were collected, pooled, and lyophilized.

⁶⁸Ga, ¹⁷⁷Lu and ²²⁵Ac Radiolabeling

⁶⁸Ga was eluted from a ⁶⁸Ge-generator with 5 mL of 0.05M HCl, collected into 2.5 mL of 12M HCl and trapped onto a DGA resin. The resin was then washed with 3 mL of 5M HCl and the purified [⁶⁸Ga]³⁺ was eluted. ¹⁷⁷Lu and ²²⁵Ac were purchased from ITM (Germany). ⁶⁸Ga, ¹⁷⁷Lu or ²²⁵Ac was added to 700 μL of 2M HEPES buffer containing 15-25 nmol of peptide. The reaction mixtures were then either heated at 85-90° C. for 15-30 min or microwaved for 1 min. For ⁶⁸Ga and ¹⁷⁷Lu labeling, each solution was purified by semi-prep HPLC followed by C18 Sep-pak purification. A radiochemical purity of >95%, determined by analytical HPLC, was required for animal studies. For ²²⁵Ac labeling, only Sep-pak purification was performed. A radiochemical purity of >95%, determined by radio-TLC, was required for animal studies.

### Cell Culture and Tumour Inoculation

LNCaP cell line was obtained from ATCC (LNCaP clone FGC, CRL-1740). It was established from a metastatic site of left supraclavicular lymph node of human prostatic adenocarcinoma. Cells were cultured in PRMI 1640 medium supplemented with 10% FBS, penicillin (100 U/mL) and streptomycin (100 μg/mL) at 37° C. in a humidified incubator containing 5% CO₂. Cells grown to 80-90% confluence were then washed with sterile phosphate-buffered saline (1×PBS pH 7.4) and trypsinization. The collected cells number was counted with a Hausser Scientific (Horsham, PA) Hemacytometer. Approximately 10 million LNCaP cells were inoculated into the left dorsal flank of immunocomprised NRG mice. The tumours were allowed to grow for 4-6 weeks and used for imaging and biodistribution studies when a volume of 200-600 mm³ was reached.

### In Vitro PSMA Competitive Binding Assay and Human Serum Albumin Binding Assay

Inhibition constants (Ki) to PSMA were measured by in vitro competition binding assays using [¹⁸F]DCFPyL as the radioligand. LNCaP cells which were plated onto a 24-well poly-D-lysine coated plate for 48 h (400,000/well). Growth medium was removed and replaced with HEPES buffered saline (50 mM HEPES, pH 7.5, 0.9% sodium chloride). After 1 h, [¹⁸F]DCFPyL (0.1 nM) was added to each well (in triplicate) containing varied concentrations (0.5 mM-0.05 nM) of tested compounds The assay mixtures were incubated for 1 h at 37° C. with gentle agitation followed by two washes with cold HEPES buffered saline. A trypsin solution (0.25%, 400 μL) was then added to each well to harvest the cells. Radioactivity was measured by gamma counting and Ki values calculated using the ‘one site—fit Ki’ built-in model in Prism 8 (GraphPad).

Human serum albumin binding assay was performed using Transil HSA binding kit (Sovicell) according to manufacturer's recommended procedures.

### PET/CT Imaging, SPECT/CT and Biodistribution

PET imaging experiments were conducted using Siemens Inveon micro PET/CT scanner. SPECT imaging experiments were conducted using an MILabs micro SPECT/CT scanner. Each tumor bearing mouse was injected 4-6 MBq of ⁶⁸Ga or 18.5 MBq of ¹⁷⁷Lu labeled tracer through the tail vein under anesthesia (2% isoflurane in oxygen). The mice were allowed to recover and roam freely in their cage. At pre-defined time point, the mice were sedated again with 2% isoflurane in oxygen inhalation and positioned in the scanner. A 10-min CT scan was conducted first for localization and attenuation correction after segmentation for reconstructing the PET or SPECT images. Then, a 10-min static PET imaging or a one hour (30 min×2 frames) of static SPECT scan was performed to determined uptake in tumor and other organs. The mice were kept warm by a heating pad during acquisition.

For biodistribution studies, the mice were injected with the radiotracer as described above. The mice were anesthetized with 2% isoflurane inhalation, and euthanized by CO₂ inhalation. Blood was withdrawn immediately from the heart, and the organs/tissues of interest were collected. The collected organs/tissues were weighed and counted using a Perkin Elmer (Waltham, MA) Wizard2 2480 gamma counter. The uptake in each organ/tissue was normalized to the injected dose (radioactivity) using a standard curve, and expressed as the percentage of the injected dose per gram of tissue (% ID/g).

### Example 1: Synthesis and Evaluation of Ccz02009 in Comparison to HTK03170 without a Gly Spacer

The chemical structure of CCZ02009 is below:

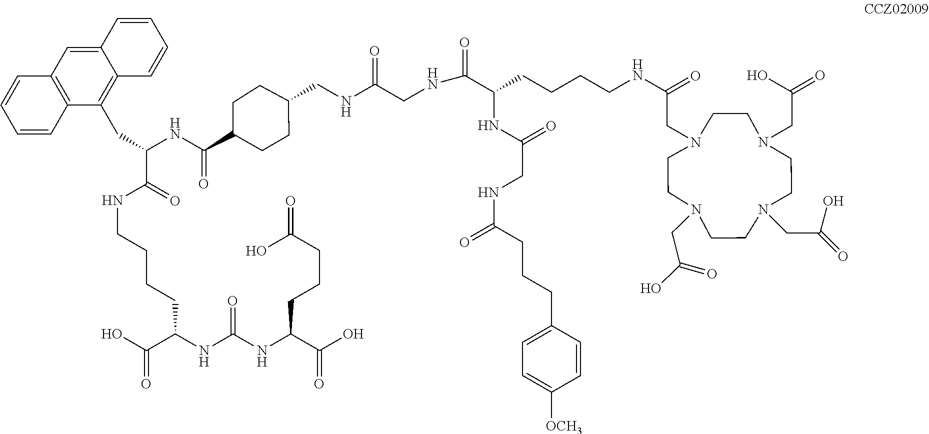

The synthesis of CCZ02009 follows the general synthesis procedures as described above except that CCZ02009 is based on the Lys-ureido-Aad moiety. To generate the isocyanate of the 2-aminoadipyl moiety, a solution of L-2-aminoadipic acid (Aad) di-tertbutyl ester hydrochloride and diisopropylethylamine in CH₂Cl₂ was cooled to −78° C. in a dry ice/acetone bath. Triphosgene was dissolved in CH₂Cl₂, and the resulting solution was added dropwise to the reaction at −78° C. The reaction was then allowed to warm to room temperature and stirred for 30 minutes to give a solution of the isocyanate of the 2-aminoadipyl moiety. Following the urea formation and ivDde deprotection on the Lys side chain, Fmoc-Ala(9-anth)-OH, Fmoc-Tranexamic acid, Fmoc-Gly-OH, Fmoc-Lys(ivDde)-OH, Fmoc-Gly-OH and 4-(p-methoxyphenyl)butyric acid were coupled sequentially. Subsequently, the ivDde group was deprotected and finally DOTA-tris(t-bu)ester was coupled. CCZ02009 was then purified by HPLC. Mass calculated [M+2H]²⁺=762.9, found 763.0.

The chemical structure of HTK03170 is shown below:

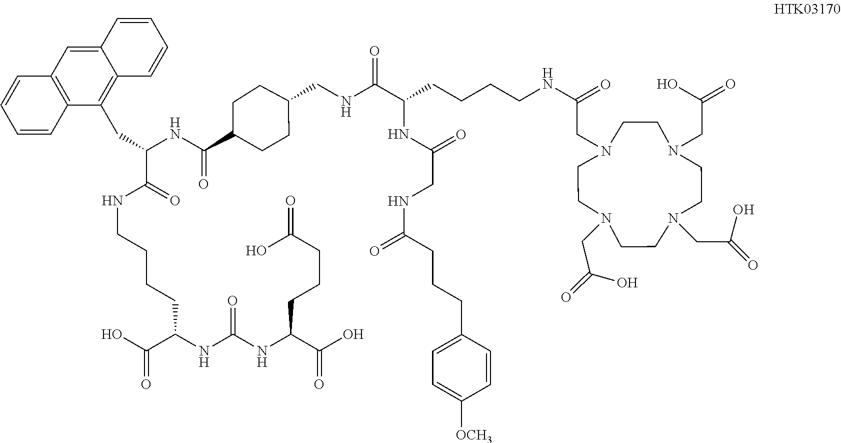

As a comparison, HTK03170 was synthesized, which lacks a glycine spacer between the PSMA and albumin binding moieties shown in CCZ02009. Mass calculated [M+2H]²⁺=734.4, found 734.7.

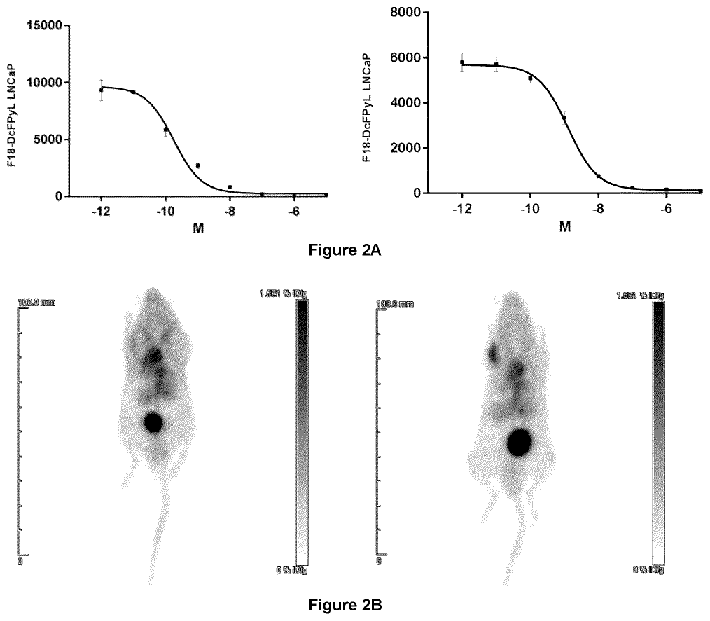

Table 3 shows that with a Gly spacer between the PSMA and albumin binding moieties, PSMA binding affinity increased from 1.53±0.33 to 0.12±0.02 nM, and albumin binding affinity increased from 70.1±3.2 to 64.9±2.2 μM. FIG. 2A shows the representative binding affinity curves for CCZ02009 (left panel) and HTK03170 (right panel). FIG. 2B and Table 4 show the PET imaging and biodistribution results using ⁶⁸Ga-CCZ02009 in NRG-mice bearing LNCaP tumors 1 h (FIG. 2B left panel) and 3 h (FIG. 2B right panel) post-injection (p.i.). Overall high and sustained blood radioactivity was observed at both timepoints, indicating the strong albumin binding of CCZ02009.

TABLE 3
PSMA and human albumin binding affinities
for CCZ02009 and HTK03170 (n = 3).

| | PSMA binding affinity | Human albumin binding affinity |
|---|---|---|
| | (Ki, nM) | (Kd, μM) |
| CCZ02009 | 0.12 ± 0.02 | 64.9 ± 2.2 |
| HTK03170 | 1.53 ± 0.33 | 70.1 ± 3.2 |

TABLE 4
Biodistribution results in NRG-mice bearing LNCaP tumors
administered with ⁶⁸Ga-CCZ02009 at 1 and 3 h p.i. Values
are in percent injected dose per gram of tissue (% ID/g).

| | |
|---|---|
| 1 h p.i. | 3 h p.i. |
| (n = 4) | (n = 4) |

| ⁶⁸Ga-CCZ02009 | Avg | Std | Avg | Std |
|---|---|---|---|---|
| Blood | 19.52 | 4.03 | 19.18 | 2.33 |
| Urine | 63.67 | 19.48 | 56.09 | 42.58 |
| Fat | 1.35 | 0.14 | 1.52 | 0.52 |
| Seminal gland | 0.88 | 0.13 | 1.73 | 1.04 |
| Testes | 2.33 | 0.48 | 2.28 | 0.83 |
| Intestine | 1.72 | 0.41 | 1.53 | 0.28 |
| Spleen | 1.56 | 0.12 | 2.17 | 0.93 |
| Pancreas | 1.56 | 0.36 | 1.41 | 1.03 |
| Stomach | 0.71 | 0.25 | 0.73 | 0.23 |
| Liver | 3.04 | 0.49 | 3.35 | 0.83 |
| Adrenal gland | 3.82 | 1.77 | 4.34 | 1.50 |
| kidney | 7.57 | 2.09 | 9.28 | 2.06 |
| Heart | 4.02 | 0.91 | 3.76 | 0.36 |
| Lungs | 6.51 | 0.71 | 7.15 | 1.78 |
| LNCaP tumor | 7.11 | 0.95 | 17.69 | 4.44 |
| Bone | 0.77 | 0.13 | 1.62 | 0.61 |
| Muscle | 1.15 | 0.11 | 1.34 | 0.27 |
| Brain | 0.30 | 0.08 | 0.32 | 0.08 |
| Salivary gland | 2.74 | 0.52 | 3.82 | 1.16 |
| Thyroid | 2.92 | 0.51 | 3.59 | 0.73 |

### Example 2: Synthesis and Evaluation of CCZ02060 and CCZ02059 with Additional Gly Spacers

The chemical structures of CCZ02060 and CCZ02059 are shown below:

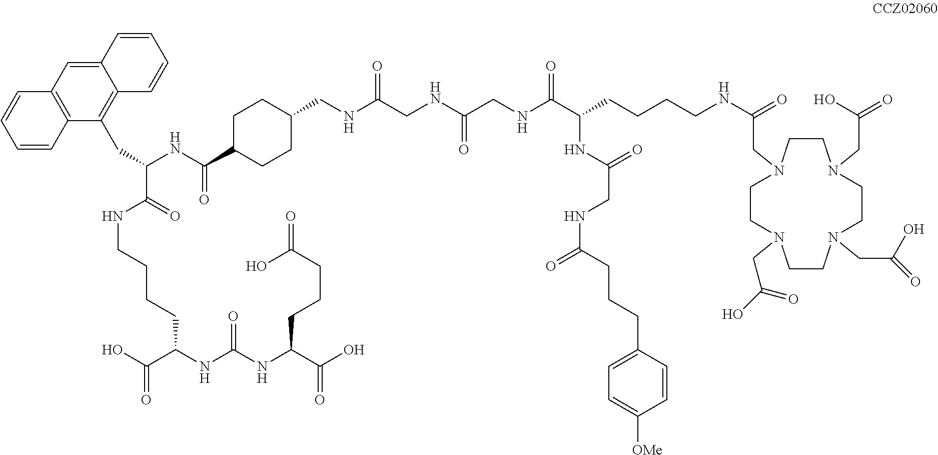

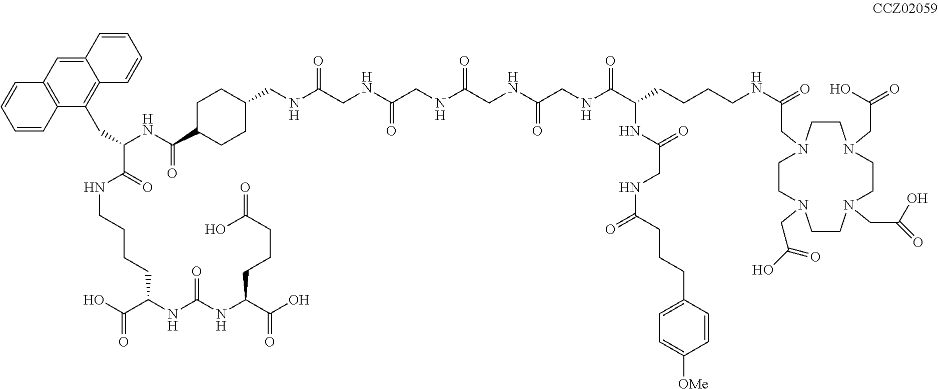

The synthesis of CCZ02060 and CCZ02059 follows the synthesis procedures of CCZ02009 as described above, except that CCZ02060 contains two Gly spacers and CCZ02059 contains four Gly spacers. For CCZ02060, mass calculated [M+2H]²⁺=791.4, found 791.5. For CCZ02059, mass calculated [M+2H]²⁺=848.4, found 848.5.

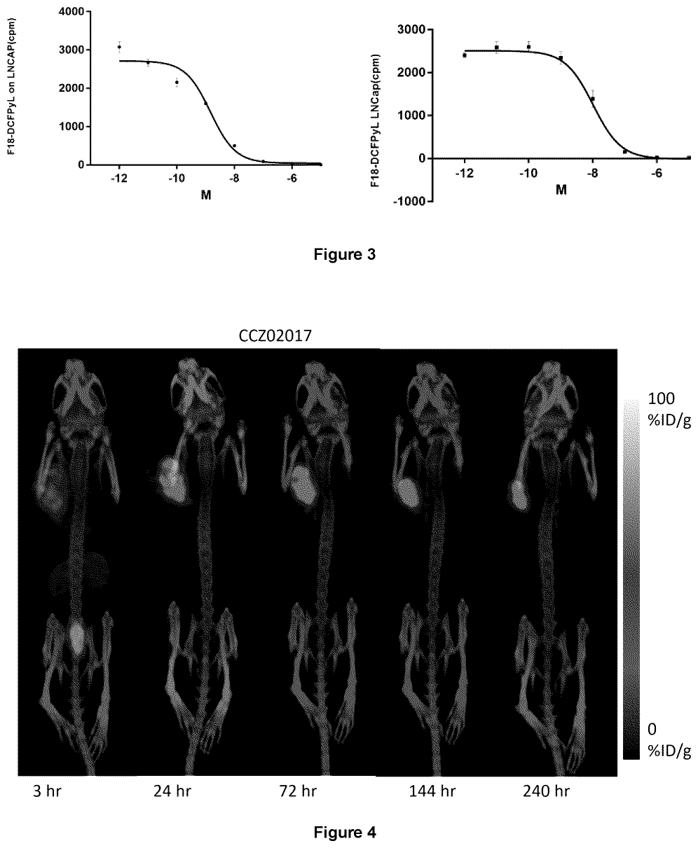

CCZ02060 and CCZ02059 binds to PSMA with affinities (Ki) of 1.09±0.19 nM and 10.84±3.28 nM (n=2), respectively. FIG. 3 shows the representative binding affinity curves for CCZ02060 (left panel) and CCZ02059 (right panel). The number of Gly spacers has an impact on the PSMA binding affinity, i.e. the most favorable binding affinity was observed with a single Gly spacer (CCZ02009), followed by two Gly spacers (CCZ02060) and four Gly spacers (CCZ02059).

### Example 3: Synthesis and Evaluation of CCZ02017 in Comparison to HTK03170 without a Gly Spacer

The chemical structure of CCZ02017 is shown below:

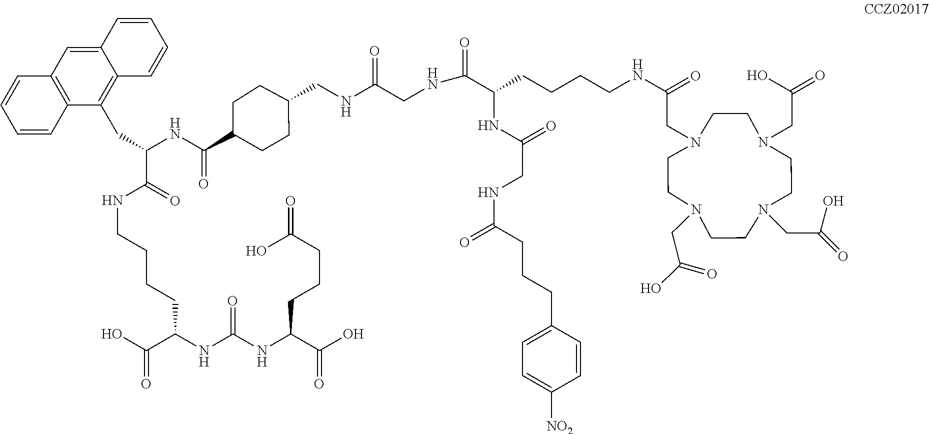

The synthesis of CCZ02017 follows the synthesis procedures of CCZ02009 as described above, except for the albumin binder, which is 4-(p-nitrophenyl)butyric acid in CCZ02017. The 4-(p-nitrophenyl)butyric acid is substantially weaker than 4-(p-methoxyphenyl)butyric acid (Ref. Kuo et al. J Nucl Med. 2021 April; 62(4):521-527). FIG. 4 shows SPECT/CT images of ¹⁷⁷Lu-CCZ02017 in NRG-mice bearing LNCaP tumors at 3, 24, 72, 144 and 240 h p.i. Table 5 shows the biodistribution of ¹⁷⁷Lu-CCZ02017 in NRG-mice bearing LNCaP tumors at 3, 24, 72, 144 and 240 h p.i. In comparison, ¹⁷⁷Lu-HTK03170 shows similar blood % ID/g values (Table 4) to ¹⁷⁷Lu-CCZ02017 at 24 and 72 h p.i. (see Table 6). Since CCZ02017 has a weaker albumin binder, the data indicates that the introduction of a Gly spacer improves albumin binding. The LNCaP tumor uptake in ¹⁷⁷Lu-CCZ02017 is substantially improved compared to ¹⁷⁷Lu-HTK03170.

TABLE 5
Biodistribution results in NRG-mice bearing LNCaP tumors administered with
¹⁷⁷Lu-CCZ02017 at 3, 24, 72, 144 and 240 h p.i. Values are in % ID/g.

| | 3 h p.i. | | 24 h p.i. | | 72 h p.i. | | 144 h p.i. | | 240 h p.i. | |
|---|---|---|---|---|---|---|---|---|---|---|
| ¹⁷⁷Lu- | (n = 5) | | (n = 5) | | (n = 5) | | (n = 5) | | (n = 5) | |
| CCZ02017 | Avg | Std | Avg | Std | Avg | Sto | Avg | Std | Avg | Std |
| Blood | 14.17 | 0.60 | 0.61 | 0.02 | 0.07 | 0.03 | 0.01 | 0.00 | 0.00 | 0.00 |
| Urine | 97.91 | 47.95 | 9.85 | 3.59 | 2.63 | 0.98 | 1.88 | 0.85 | 0.43 | 0.24 |
| Fat | 1.85 | 0.24 | 0.20 | 0.05 | 0.15 | 0.04 | 0.22 | 0.13 | 0.06 | 0.03 |
| Seminal | 0.93 | 0.13 | 0.07 | 0.02 | 0.03 | 0.00 | 0.01 | 0.01 | 0.00 | 0.00 |
| gland | | | | | | | | | | |
| Testes | 6.56 | 7.05 | 0.48 | 0.07 | 0.29 | 0.03 | 0.15 | 0.03 | 0.08 | 0.01 |
| Intestine | 1.03 | 0.56 | 0.13 | 0.02 | 0.04 | 0.01 | 0.01 | 0.00 | 0.01 | 0.00 |
| Spleen | 2.25 | 1.09 | 0.35 | 0.07 | 0.38 | 0.08 | 0.37 | 0.17 | 0.50 | 0.51 |
| Pancreas | 2.27 | 1.61 | 0.11 | 0.02 | 0.04 | 0.01 | 0.01 | 0.01 | 0.00 | 0.00 |
| Stomach | 1.55 | 1.84 | 0.15 | 0.02 | 0.04 | 0.02 | 0.02 | 0.02 | 0.01 | 0.01 |
| Liver | 2.31 | 1.13 | 0.31 | 0.04 | 0.23 | 0.11 | 0.09 | 0.02 | 0.06 | 0.05 |
| Adrenal | 3.41 | 0.69 | 0.61 | 0.08 | 0.34 | 0.12 | 0.23 | 0.08 | 0.02 | 0.02 |
| gland | | | | | | | | | | |
| kidney | 17.31 | 1.01 | 5.58 | 0.59 | 3.21 | 0.84 | 1.98 | 0.61 | 0.46 | 0.09 |
| Heart | 3.24 | 0.24 | 0.24 | 0.07 | 0.10 | 0.02 | 0.04 | 0.02 | 0.01 | 0.01 |
| Lungs | 6.68 | 0.69 | 0.55 | 0.03 | 0.16 | 0.03 | 0.08 | 0.03 | 0.14 | 0.27 |
| LNCaP | 39.27 | 8.72 | 89.90 | 30.12 | 70.84 | 21.79 | 116.43 | 43.13 | 95.45 | 15.98 |
| tumor | | | | | | | | | | |
| Bone | 0.78 | 0.13 | 0.07 | 0.03 | 0.02 | 0.00 | 0.02 | 0.02 | 0.01 | 0.02 |
| Muscle | 1.18 | 0.08 | 0.08 | 0.01 | 0.02 | 0.01 | 0.01 | 0.01 | 0.01 | 0.02 |
| Brain | 0.24 | 0.02 | 0.03 | 0.00 | 0.01 | 0.00 | 0.01 | 0.00 | 0.00 | 0.00 |
| Salivary | 2.43 | 0.85 | 0.35 | 0.03 | 0.14 | 0.03 | 0.05 | 0.03 | 0.01 | 0.01 |
| gland | | | | | | | | | | |
| Thyroid | 2.87 | 0.18 | 0.40 | 0.05 | 0.16 | 0.09 | 0.11 | 0.02 | 0.03 | 0.00 |
| Lacrimal | 0.20 | 0.06 | 0.04 | 0.06 | 0.01 | 0.02 | 0.00 | 0.00 | 0.00 | 0.00 |
| gland | | | | | | | | | | |

TABLE 6
Biodistribution results in NRG-mice bearing
LNCaP tumors administered with ¹⁷⁷Lu-HTK03170
at 24 and 72 h p.i. Values are in % ID/g.

| | |
|---|---|
| 24 h p.i. | 72 h p.i. |
| (n = 5) | (n = 5) |

| ¹⁷⁷Lu-HTK03170 | Avg | Std | Avg | Std |
|---|---|---|---|---|
| Blood | 0.73 | 0.21 | 0.07 | 0.01 |
| Urine | 19.02 | 13.28 | 4.96 | 1.96 |
| Intestine | 0.18 | 0.05 | 0.08 | 0.04 |
| Spleen | 0.33 | 0.07 | 0.36 | 0.12 |
| Liver | 0.49 | 0.05 | 0.27 | 0.02 |
| kidney | 6.06 | 0.91 | 4.30 | 0.84 |
| LNCaP tumor | 42.96 | 6.66 | 56.98 | 5.98 |
| Bone | 0.18 | 0.11 | 0.19 | 0.08 |
| Muscle | 0.10 | 0.01 | 0.15 | 0.07 |
| Salivary gland | 0.35 | 0.08 | 0.31 | 0.09 |

TABLE 7
Biodistribution results in NRG-mice bearing LNCaP tumors administered with
²²⁵Ac-CCZ02017 at 3, 24, 72, 144 and 240 h p.i. Values are in % ID/g.

| | 3 h p.i. | | 24 h p.i. | | 72 h p.i. | | 144 h p.i. | | 240 h p.i. | |
|---|---|---|---|---|---|---|---|---|---|---|
| | (n = 5) | | (n = 5) | | (n = 5) | | (n = 5) | | (n = 5) | |
| ²²⁵Ac-CCZ02017 | Avg | Std | Avg | Std | Avg | Std | Avg | Std | Avg | Std |
| Blood | 9.83 | 1.34 | 0.28 | 0.16 | 0.01 | 0.01 | 0.01 | 0.01 | 0.02 | 0.02 |
| Urine | 142.80 | 26.10 | 4.30 | 3.43 | 1.33 | 1.30 | 0.55 | 0.73 | 0.41 | 0.50 |
| Fat | 1.96 | 0.60 | 0.13 | 0.02 | 0.40 | 0.63 | 0.07 | 0.01 | 0.13 | 0.05 |
| Seminal gland | 2.26 | 3.64 | 0.15 | 0.15 | 0.03 | 0.02 | 0.04 | 0.01 | 0.06 | 0.02 |
| Testes | 2.73 | 0.62 | 0.38 | 0.14 | 0.33 | 0.18 | 0.29 | 0.11 | 0.27 | 0.04 |
| Intestine | 1.02 | 0.13 | 0.15 | 0.08 | 0.06 | 0.02 | 0.06 | 0.04 | 0.06 | 0.02 |
| Spleen | 1.15 | 0.49 | 0.50 | 0.14 | 0.93 | 0.87 | 0.65 | 0.24 | 1.14 | 0.40 |
| Pancreas | 1.14 | 0.10 | 0.09 | 0.01 | 0.09 | 0.04 | 0.14 | 0.18 | 0.06 | 0.02 |
| Stomach | 0.47 | 0.12 | 0.20 | 0.13 | 0.06 | 0.03 | 0.05 | 0.03 | 0.08 | 0.03 |
| Liver | 1.90 | 0.12 | 0.68 | 0.16 | 0.51 | 0.26 | 0.71 | 0.12 | 1.49 | 0.33 |
| Adrenal gland | 3.61 | 1.96 | 0.59 | 0.24 | 24.11 | 49.82 | 0.31 | 0.24 | 0.53 | 0.21 |
| kidney | 14.29 | 2.18 | 6.87 | 2.16 | 3.62 | 0.56 | 1.95 | 0.43 | 2.24 | 0.65 |
| Heart | 2.27 | 0.10 | 0.25 | 0.08 | 0.16 | 0.05 | 0.14 | 0.06 | 0.16 | 0.06 |
| Lungs | 4.93 | 0.63 | 0.58 | 0.13 | 0.19 | 0.04 | 0.13 | 0.05 | 0.18 | 0.03 |
| LNCaP tumor | 23.63 | 2.08 | 42.31 | 9.38 | 54.12 | 15.76 | 71.25 | 8.78 | 123.67 | 15.35 |
| Bone | 1.24 | 0.74 | 0.30 | 0.10 | 0.34 | 0.11 | 0.23 | 0.04 | 0.57 | 0.17 |
| Muscle | 0.80 | 0.12 | 0.06 | 0.02 | 0.04 | 0.01 | 0.02 | 0.01 | 0.08 | 0.09 |
| Brain | 0.19 | 0.03 | 0.03 | 0.02 | 0.02 | 0.01 | 0.02 | 0.00 | 0.02 | 0.01 |
| Salivary gland | 3.37 | 0.99 | 0.35 | 0.12 | 0.33 | 0.19 | 0.21 | 0.06 | 0.32 | 0.28 |
| Thyroid | 1.72 | 0.78 | 0.33 | 0.10 | 0.22 | 0.10 | 0.23 | 0.06 | 0.23 | 0.12 |
| Lacrimal gland | 0.92 | 0.29 | 0.32 | 0.24 | 1.39 | 1.76 | 0.65 | 0.58 | 0.19 | 0.30 |

### Example 4: Synthesis and Evaluation of CCZ02008

The chemical structure of CCZ02008 is shown below:

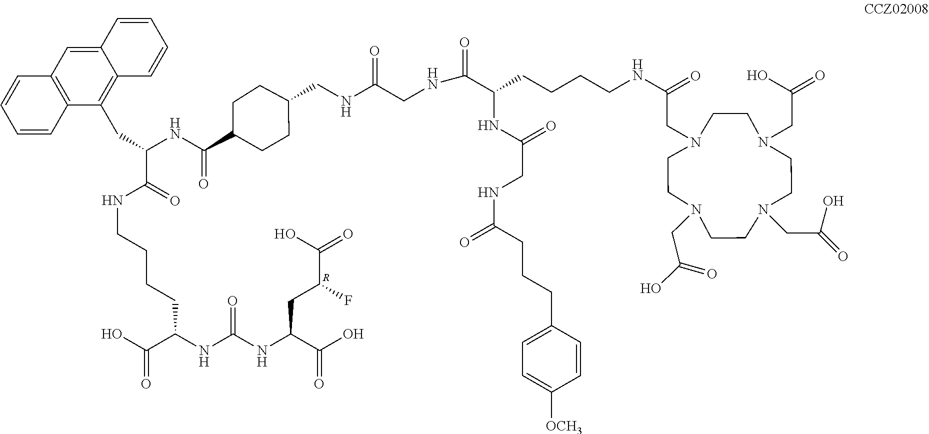

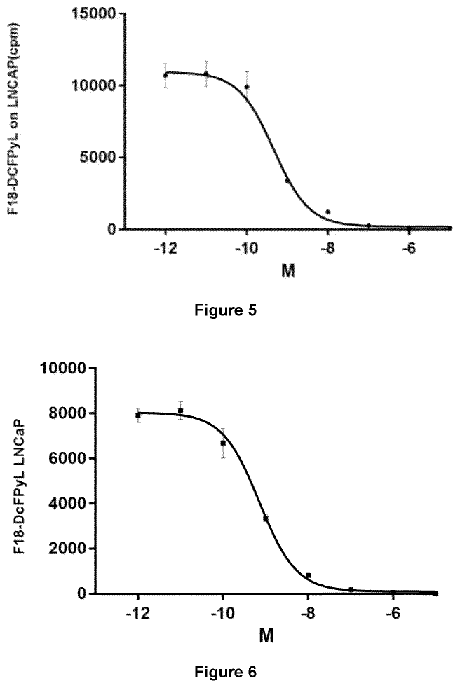

The synthesis of CCZ02008 follows the synthesis procedures of CCZ02009 as described above, and instead of Aad, it incorporates 4R-F-Glu. Mass calculated [M+2H]²⁺=764.9, found 765.2. CCZ02008 binds to PSMA with high affinity Ki=0.48±0.1 nM (n=3). See FIG. 5 and Table 8.

TABLE 8
Biodistribution results in NRG-mice bearing LNCaP
tumors administered with ¹⁷⁷Lu-CCZ02008 at 4,
24, 72 and 120 h p.i. Values are in % ID/g.

| | 4 h p.i. | | 24 h p.i. | | 72 h p.i. | | 120 h p.i. | |
|---|---|---|---|---|---|---|---|---|
| ¹⁷⁷Lu- | (n = 5) | | (n = 5) | | (n = 5) | | (n = 5) | |
| CCZ02008 | Avg | Std | Avg | Std | Avg | Std | Avg | Std |
| Blood | 24.97 | 1.63 | 4.37 | 0.93 | 0.42 | 0.20 | 0.14 | 0.03 |
| Urine | 76.61 | 24.30 | 28.57 | 11.80 | 4.51 | 2.97 | 3.80 | 2.05 |
| Fat | 2.63 | 0.55 | 0.71 | 0.15 | 0.39 | 0.17 | 0.22 | 0.06 |
| Seminal gland | 1.53 | 0.53 | 0.38 | 0.11 | 0.15 | 0.05 | 0.27 | 0.32 |
| Testes | 3.83 | 0.51 | 1.86 | 0.29 | 0.92 | 0.16 | 0.67 | 0.03 |
| Intestine | 1.87 | 0.11 | 0.59 | 0.11 | 0.15 | 0.06 | 0.14 | 0.04 |
| Spleen | 2.15 | 0.20 | 0.86 | 0.22 | 0.83 | 0.31 | 1.45 | 0.34 |
| Pancreas | 2.20 | 0.25 | 0.62 | 0.10 | 0.17 | 0.06 | 0.11 | 0.02 |
| Stomach | 1.11 | 0.05 | 0.48 | 0.13 | 0.18 | 0.12 | 0.37 | 0.20 |
| Liver | 4.26 | 0.43 | 1.16 | 0.16 | 0.47 | 0.13 | 0.30 | 0.07 |
| Adrenal gland | 5.91 | 2.67 | 2.39 | 0.60 | 2.59 | 0.98 | 1.39 | 0.64 |
| kidney | 10.56 | 1.95 | 7.55 | 1.46 | 3.53 | 1.13 | 2.04 | 0.69 |
| Heart | 5.42 | 0.45 | 1.27 | 0.21 | 0.37 | 0.07 | 0.22 | 0.02 |
| Lungs | 10.19 | 1.59 | 2.49 | 0.57 | 0.69 | 0.19 | 0.30 | 0.05 |
| LNCaP tumor | 21.30 | 1.74 | 38.57 | 5.32 | 38.46 | 4.17 | 45.01 | 6.28 |
| Bone | 1.49 | 0.20 | 0.36 | 0.09 | 0.26 | 0.18 | 0.14 | 0.11 |
| Muscle | 1.75 | 0.18 | 0.40 | 0.09 | 0.11 | 0.08 | 0.09 | 0.09 |
| Brain | 0.47 | 0.05 | 0.09 | 0.02 | 0.03 | 0.02 | 0.02 | 0.01 |
| Salivary gland | 4.23 | 0.39 | 1.38 | 0.29 | 0.55 | 0.29 | 0.34 | 0.13 |
| Thyroid | 4.29 | 0.40 | 1.54 | 0.31 | 0.65 | 0.26 | 0.39 | 0.07 |
| Lacrimal gland | 1.44 | 0.98 | 0.12 | 0.08 | 0.07 | 0.04 | 0.12 | 0.09 |

### Example 5: Synthesis and Evaluation of CCZ02025

The chemical structure of CCZ02025 is shown below:

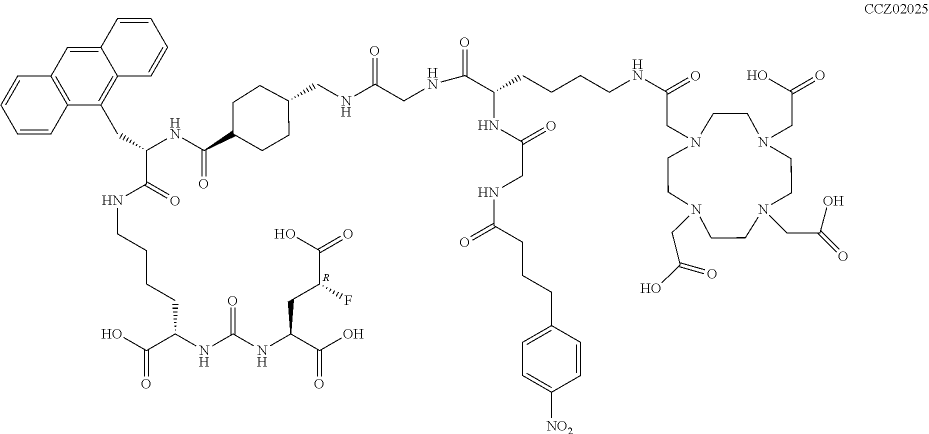

The synthesis of CCZ02025 follows the synthesis procedures of CCZ02009 as described above, instead of Aad, it incorporates 4R-F-Glu, and instead of 4-(p-methoxyphenyl)butyric acid, it incorporates 4-(p-nitrophenyl)butyric acid. Mass calculated [M+2H]²⁺=772.4, found 772.6. CCZ02025 binds to PSMA with high affinity Ki=0.59 nM (FIG. 6, n=1), and ⁶⁸Ga-CCZ02025 shows good tumor uptake and overall background tissue clearance (Table 9).

TABLE 9
Biodistribution results in NRG-mice bearing LNCaP tumors administered
with ⁶⁸Ga-CCZ02025 at 1 and 3 h p.i. Values are in % ID/g.

| | |
|---|---|
| 1 h p.i. | 3 h p.i. |
| (n = 4) | (n = 4) |

| ⁶⁸Ga-CCZ02025 | Avg | Std | Avg | Std |
|---|---|---|---|---|
| Blood | 19.40 | 1.41 | 13.83 | 0.35 |
| Urine | 104.40 | 66.50 | 101.55 | 41.69 |
| Fat | 2.74 | 0.99 | 1.79 | 0.87 |
| Seminal gland | 2.55 | 2.65 | 1.83 | 1.91 |
| Testes | 2.65 | 0.35 | 2.80 | 0.34 |
| Intestine | 1.93 | 0.12 | 1.12 | 0.20 |
| Spleen | 1.90 | 0.31 | 1.17 | 0.18 |
| Pancreas | 1.89 | 0.19 | 1.13 | 0.13 |
| Stomach | 0.59 | 0.09 | 0.57 | 0.10 |
| Liver | 4.07 | 1.19 | 3.21 | 0.72 |
| Adrenal gland | 5.96 | 2.10 | 4.26 | 3.79 |
| kidney | 14.01 | 2.96 | 9.90 | 1.05 |
| Heart | 4.13 | 0.49 | 2.86 | 0.93 |
| Lungs | 8.79 | 0.98 | 5.21 | 0.60 |
| LNCaP tumor | 9.19 | 0.77 | 18.79 | 2.25 |
| Bone | 1.01 | 0.32 | 1.27 | 0.35 |
| Muscle | 1.48 | 0.11 | 0.95 | 0.06 |
| Brain | 0.30 | 0.04 | 0.22 | 0.02 |
| Salivary gland | 3.24 | 0.77 | 2.47 | 0.20 |
| Thyroid | 4.11 | 0.48 | 2.41 | 0.14 |

### Example 6: Synthesis and Evaluation of CCZ02024

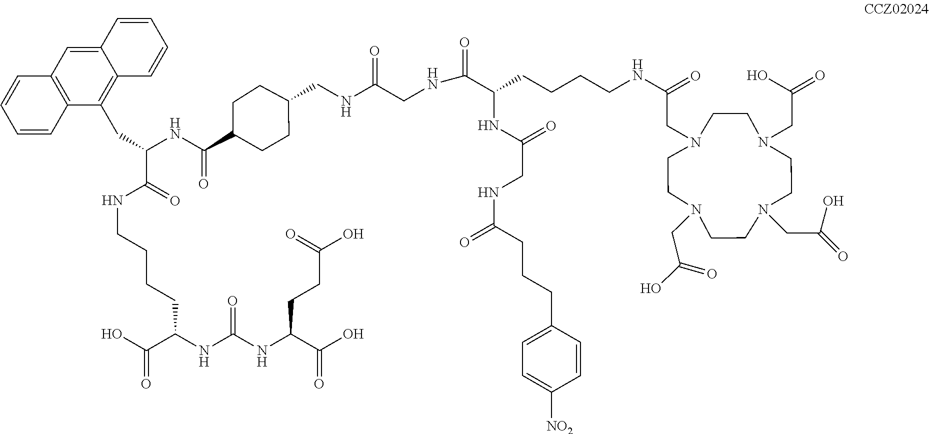

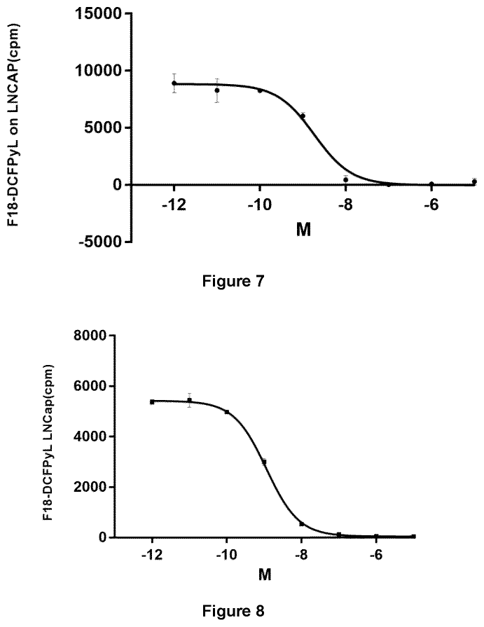

The synthesis of CCZ02024 follows the synthesis procedures of CCZ02025 as described above, instead of 4R-F-Glu, it incorporates Glu. Mass calculated [M+2H]²⁺=763.4, found 763.8. CCZ02024 binds to PSMA with high affinity Ki=1.54 nM (FIG. 7, n=1).

### Example 7: Synthesis and Evaluation of CCZ02015

The chemical structure of CCZ02015 is shown below:

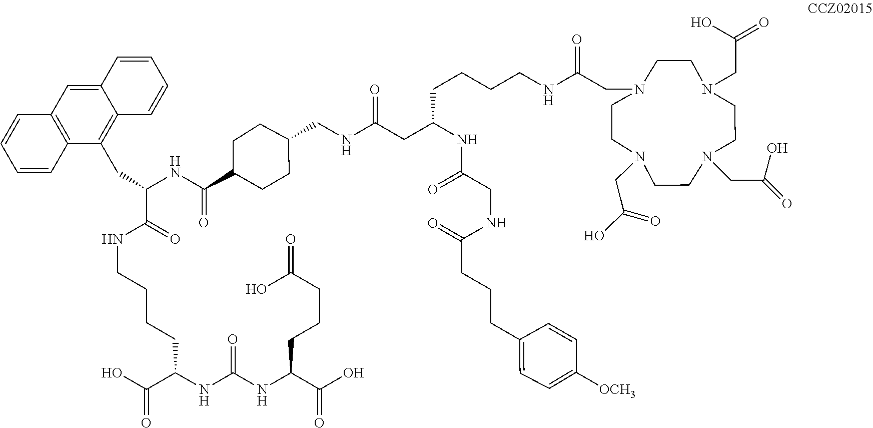

The synthesis of CCZ02015 follows the synthesis procedures of CCZ02009 as described above. The differences are that CCZ02015 does not contain the Gly spacer and incorporate an elongated linker, i.e. beta-homoLys instead of Lys. Mass calculated [M+2H]²⁺=741.4, found 741.5. CCZ02015 binds to PSMA with high affinity Ki=1.09±0.30 nM (FIG. 8, n=3), and ⁶⁸Ga-CCZ02015 shows good tumor uptake and overall background tissue clearance (Table 10).

TABLE 10
Biodistribution results in NRG-mice bearing LNCaP tumors administered
with ⁶⁸Ga-CCZ02015 at 1 and 3 h p.i. Values are in % ID/g.

| | |
|---|---|
| 1 h p.i. | 3 h p.i. |
| (n = 4) | (n = 4) |

| ⁶⁸Ga-CCZ02015 | Avg | Std | Avg | Std |
|---|---|---|---|---|
| Blood | 15.78 | 1.94 | 10.81 | 1.66 |
| Urine | 177.03 | 117.95 | 84.38 | 38.68 |
| Fat | 1.52 | 0.68 | 1.04 | 0.75 |
| Seminal gland | 0.72 | 0.17 | 1.63 | 1.36 |
| Testes | 1.77 | 0.25 | 2.07 | 1.33 |
| Intestine | 1.46 | 0.29 | 1.17 | 0.29 |
| Spleen | 1.28 | 0.24 | 1.01 | 0.28 |
| Pancreas | 1.34 | 0.20 | 1.10 | 0.29 |
| Stomach | 0.46 | 0.15 | 0.84 | 0.87 |
| Liver | 2.98 | 1.03 | 2.13 | 0.83 |
| Adrenal gland | 3.28 | 1.82 | 3.36 | 2.05 |
| kidney | 11.93 | 2.31 | 10.94 | 3.35 |
| Heart | 3.19 | 0.46 | 2.55 | 0.61 |
| Lungs | 5.90 | 1.37 | 4.65 | 1.25 |
| LNCaP tumor | 11.16 | 1.47 | 22.60 | 3.64 |
| Bone | 0.92 | 0.58 | 1.20 | 0.61 |
| Muscle | 1.07 | 0.12 | 1.13 | 0.60 |
| Brain | 0.25 | 0.04 | 0.19 | 0.06 |
| Salivary gland | 2.19 | 0.28 | 2.18 | 0.45 |
| Thyroid | 2.54 | 0.22 | 2.09 | 0.73 |

### Example 8: Synthesis and Evaluation of CCZ02019

The chemical structure of CCZ02019 is shown below:

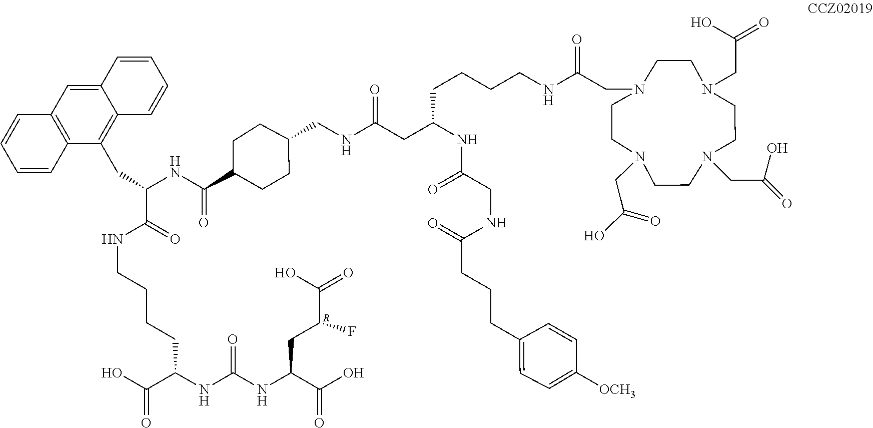

The synthesis of CCZ02019 follows the synthesis procedures of CCZ02015 as described above. The difference is that CCZ02019 incorporates 4R-F-Glu instead of Aad in the PSMA binding motif. Mass calculated [M+2H]²⁺=743.4, found 743.7. ¹⁷⁷Lu-CCZ02019 shows good tumor uptake and overall background tissue clearance (Table 11).

TABLE 11
Biodistribution results in NRG-mice bearing LNCaP tumors administered with
¹⁷⁷Lu-CCZ02019 at 3, 24, 72, 144 and 240 h p.i. Values are in % ID/g.

| | 3 h p.i. | | 24 h p.i. | | 72 h p.i. | | 144 h p.i. | | 240 h p.i. | |
|---|---|---|---|---|---|---|---|---|---|---|
| | (n = 4) | | (n = 4) | | (n = 4) | | (n = 4) | | (n = 5) | |
| ¹⁷⁷Lu-CCZ02019 | Avg | Std | Avg | Std | Avg | Std | Avg | Std | Avg | Std |
| Blood | 11.22 | 1.88 | 0.70 | 0.21 | 0.13 | 0.07 | 0.04 | 0.02 | 0.01 | 0.00 |
| Urine | 180.95 | 58.73 | 8.42 | 1.26 | 1.12 | 0.47 | 0.84 | 0.48 | 0.37 | 0.30 |
| Fat | 1.91 | 0.79 | 0.33 | 0.15 | 0.16 | 0.03 | 0.11 | 0.01 | 0.06 | 0.04 |
| Seminal gland | 0.59 | 0.13 | 0.23 | 0.25 | 0.06 | 0.02 | 0.03 | 0.01 | 0.01 | 0.01 |
| Testes | 2.55 | 0.41 | 0.37 | 0.22 | 0.39 | 0.06 | 0.33 | 0.05 | 0.16 | 0.05 |
| Intestine | 0.98 | 0.22 | 0.23 | 0.06 | 0.08 | 0.03 | 0.03 | 0.00 | 0.02 | 0.00 |
| Spleen | 1.53 | 0.62 | 0.37 | 0.08 | 0.45 | 0.17 | 0.51 | 0.08 | 0.46 | 0.34 |
| Pancreas | 1.07 | 0.19 | 0.17 | 0.04 | 0.09 | 0.02 | 0.06 | 0.01 | 0.03 | 0.01 |
| Stomach | 0.58 | 0.42 | 0.73 | 0.71 | 0.15 | 0.13 | 0.03 | 0.01 | 0.02 | 0.00 |
| Liver | 2.18 | 0.49 | 0.48 | 0.15 | 0.29 | 0.08 | 0.19 | 0.02 | 0.15 | 0.09 |
| Adrenal gland | 2.79 | 0.56 | 0.54 | 0.21 | 0.53 | 0.16 | 0.31 | 0.05 | 0.11 | 0.03 |
| kidney | 10.42 | 1.01 | 4.02 | 0.88 | 1.84 | 0.68 | 1.10 | 0.20 | 0.52 | 0.22 |
| Heart | 2.52 | 0.49 | 0.37 | 0.07 | 0.18 | 0.04 | 0.13 | 0.02 | 0.05 | 0.02 |
| Lungs | 5.22 | 1.25 | 0.65 | 0.36 | 0.41 | 0.20 | 0.93 | 1.47 | 0.09 | 0.04 |
| LNCaP tumor | 50.23 | 13.43 | 55.39 | 5.20 | 45.10 | 12.28 | 40.77 | 14.92 | 25.30 | 15.24 |
| Bone | 1.03 | 0.77 | 0.18 | 0.03 | 0.05 | 0.03 | 0.04 | 0.04 | 0.00 | 0.01 |
| Muscle | 0.87 | 0.18 | 0.11 | 0.04 | 0.04 | 0.02 | 0.03 | 0.01 | 0.01 | 0.01 |
| Brain | 0.18 | 0.04 | 0.03 | 0.00 | 0.01 | 0.00 | 0.01 | 0.00 | 0.00 | 0.00 |
| Salivary gland | 1.92 | 0.20 | 0.56 | 0.18 | 0.27 | 0.05 | 0.34 | 0.28 | 0.10 | 0.05 |
| Thyroid | 2.44 | 0.50 | 0.52 | 0.06 | 0.37 | 0.04 | 0.28 | 0.10 | 0.12 | 0.04 |
| Lacrimal gland | 0.39 | 0.22 | 0.00 | 0.00 | 0.00 | 0.00 | 0.00 | 0.00 | 0.00 | 0.00 |

### Example 9: Synthesis and Evaluation of CCZ02012 and CCZ02013

The chemical structure of CCZ02012 (left) and CCZ02013 (right) are shown below:

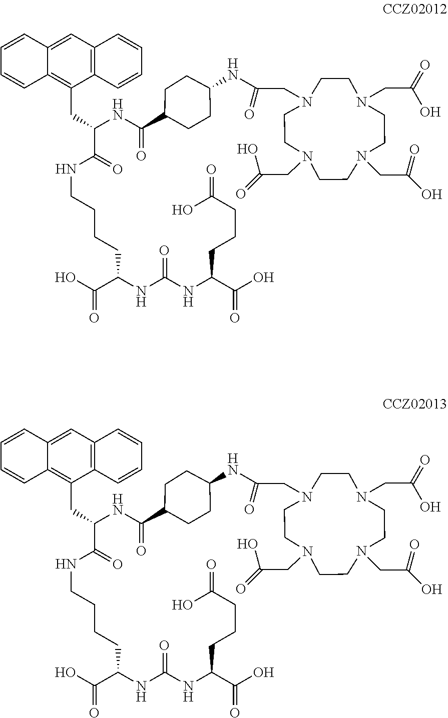

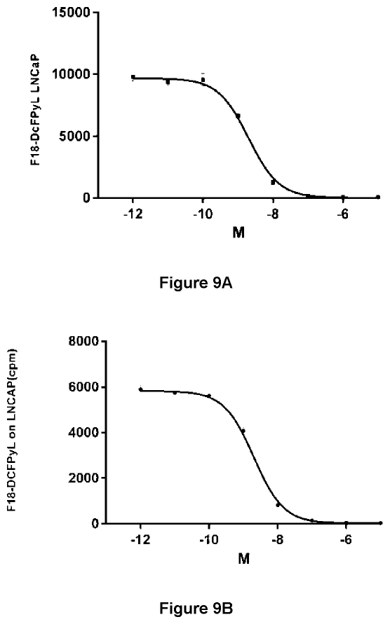

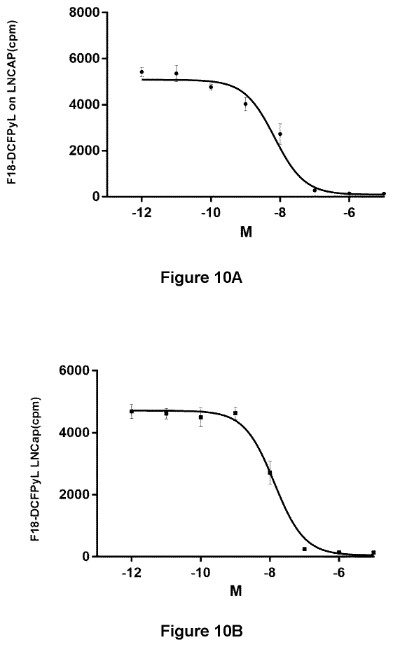

The synthesis of CCZ02012 and CCZ02013 follows the general synthesis procedures as described above on the Lys-ureido-Aad backbone, and instead of Fmoc-Tranexamic acid, Fmoc-trans-4-aminocyclohexane carboxylic acid (ACHC) and Fmoc-cis-4-ACHC were incorporated, respectively. For CCZ02012, mass calculated [M+2H]²⁺=546.8, found 547.1. For CCZ02013, mass calculated [M+2H]²⁺=546.8, found 547.2. The use of ACHC, either trans or cis, did not affect the binding to PSMA substantially. CCZ02012 and CCZ02013 bind to PSMA at Ki=1.67 nM (n=1) and Ki=1.53±0.21 nM (n=2), respectively. FIG. 9 shows the binding assay curves for CCZ02012 (9A) and CCZ02013 (9B).

### Example 10: Synthesis and Evaluation of CCZ02021 and CCZ02022

The chemical structures of CCZ02021 and CCZ02022 are shown below:

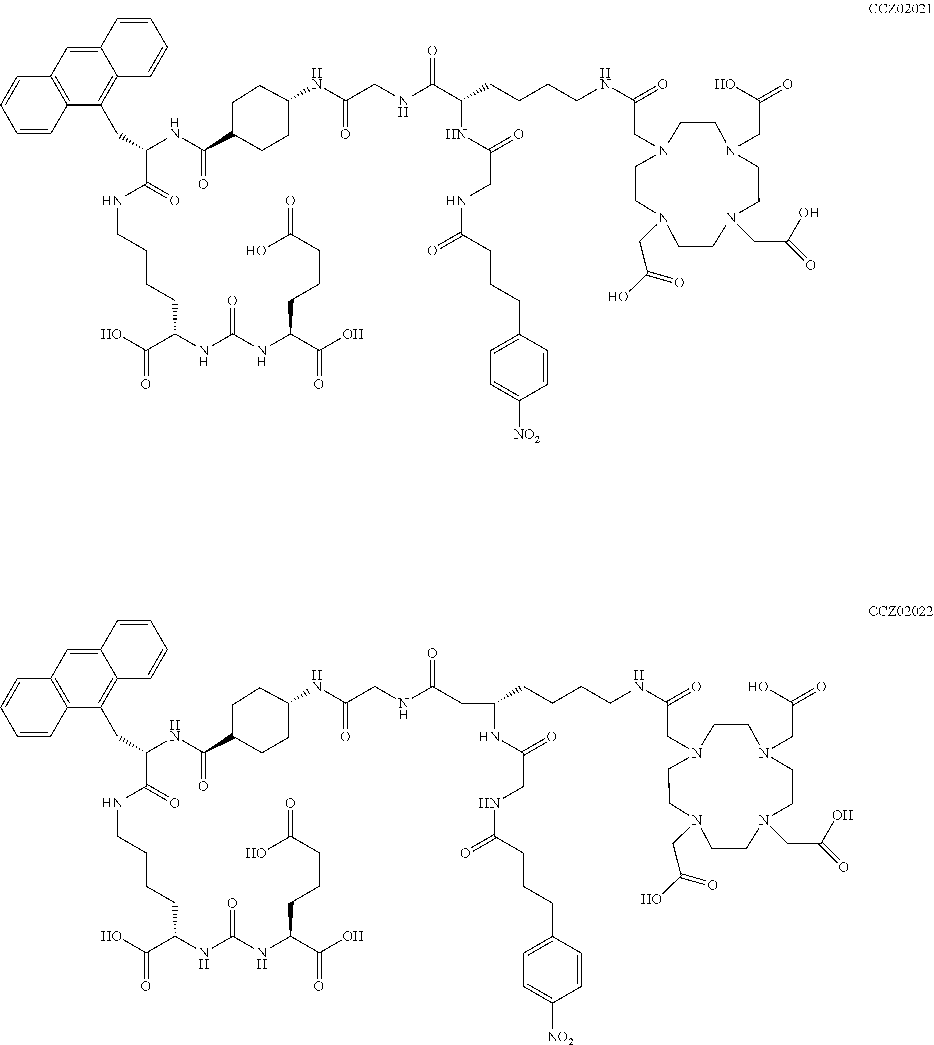

The synthesis of CCZ02021 and CCZ02022 follows the synthesis procedures of CCZ02012 as described with CCZ02021 incorporating a Gly spacer, and CCZ02022 incorporating a Gly spacer and beta-homoLys instead of Lys. For CCZ02021, mass calculated [M+2H]²⁺=763.4, found 763.5. For CCZ02022, mass calculated [M+2H]²⁺=770.4, found 770.7. CCZ02021 and CCZ02022 bind to PSMA at Ki=5.71 nM (n=1) and Ki=11.2 nM (n=1), respectively. FIG. 9 shows the binding assay curves for CCZ02021 (10A) and CCZ02022 (10B).

### Example 11: Synthesis and Evaluation of CCZ02034

The chemical structure of CCZ02034 is shown below:

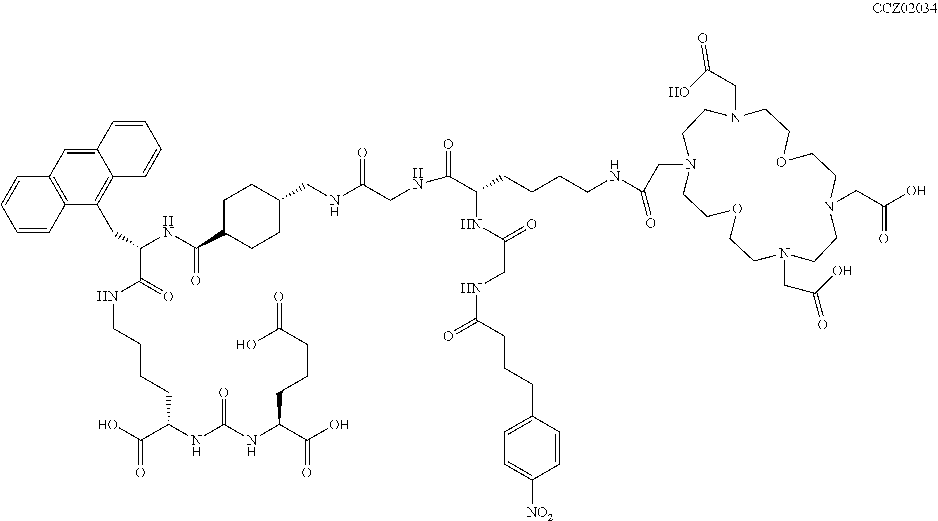

The synthesis of CCZ02034 follows the synthesis procedures of CCZ02017 as described with CCZ02034 incorporating a crown chelator instead of DOTA. Mass calculated [M+2H]²⁺=814.4, found 814.6. CCZ02034 binds to PSMA at Ki=2.82 nM (n=1). See FIG. 11.

### Example 12: Synthesis and Evaluation of CCZ02005

The chemical structure of CCZ02005 is shown below:

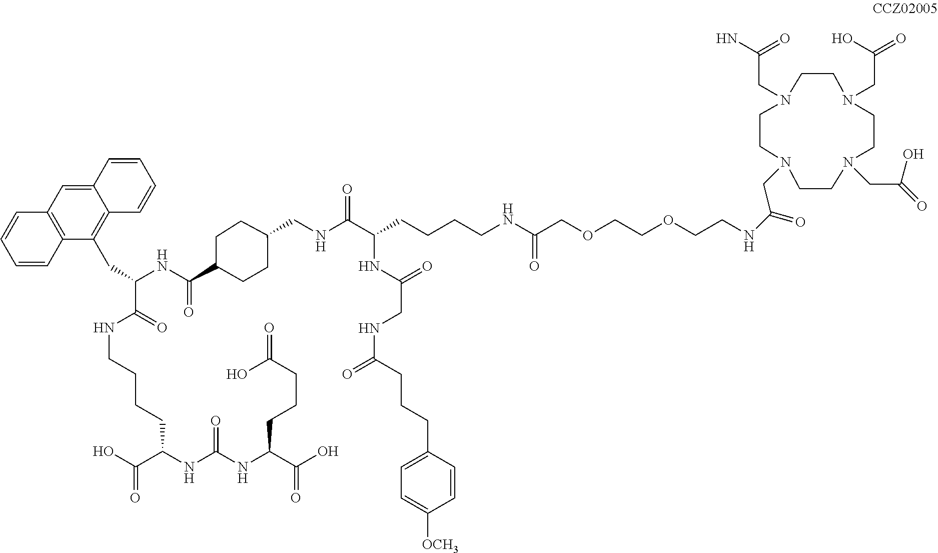

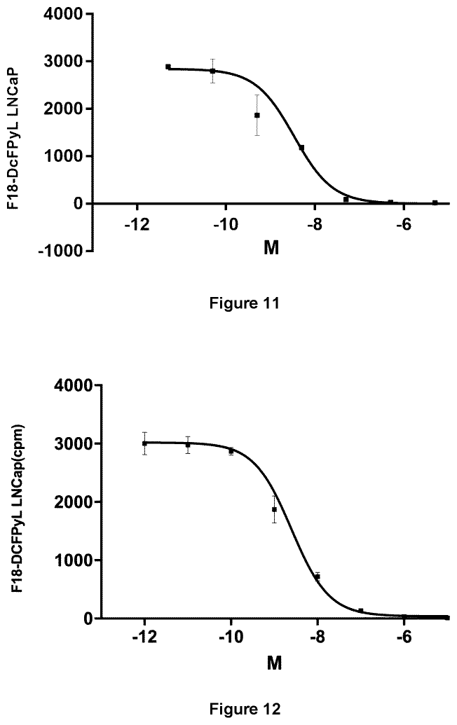

The synthesis of CCZ02005 follows the general procedures as described above with a PEG₂ spacer incorporated between the albumin binding group and the DOTA chelator. Mass calculated [M+2H]²⁺=806.9, found 807.0. CCZ02005 binds to PSMA at Ki=2.08 nM (n=1). See FIG. 12. The binding affinity is comparable to HTK03170, indicating that adding a spacer between the albumin binder and the chelator does not substantially impact the binding affinity to PSMA.

### Example 13: Synthesis and Evaluation of CCZ02061

The chemical structure of CCZ02061 is shown below:

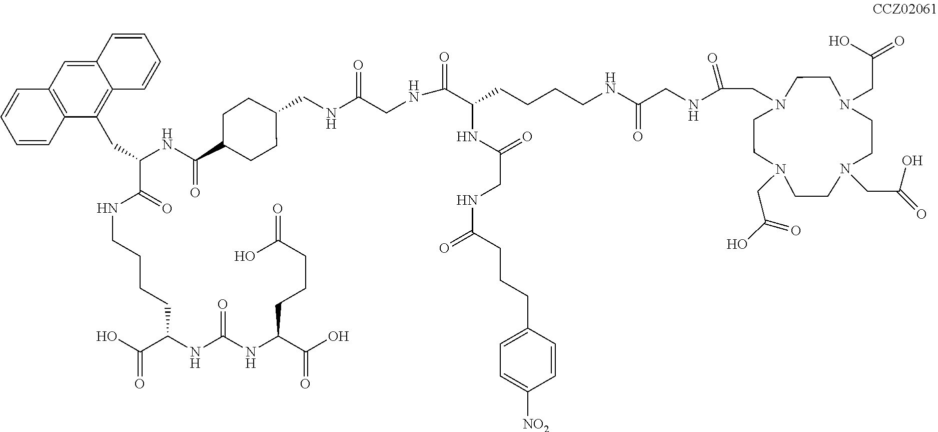

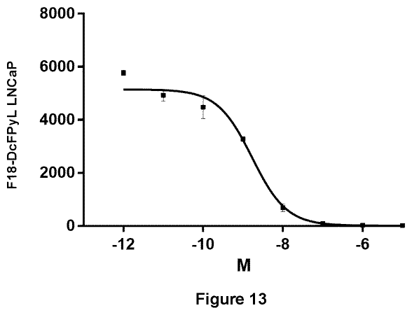

The synthesis of CCZ02061 follows the synthesis procedures of CCZ02017 described above with addition of a Gly spacer incorporated between the albumin binding group and the DOTA chelator. Mass calculated [M+2H]²⁺=798.9, found 798.9. CCZ02017 binds to PSMA at Ki=1.14±0.37 nM (n=2). See FIG. 13. The present invention has been described with regard to one or more embodiments. However, it will be apparent to persons skilled in the art that a number of variations and modifications can be made without departing from the scope of the invention as defined in the following claims. The scope of the invention should therefore not be limited by the preferred embodiments set forth in the above Examples, but should be given the broadest interpretation consistent with the description as a whole.

What is claimed is:

1. A compound, wherein the compound has Formula I or is a salt or a solvate of Formula I: 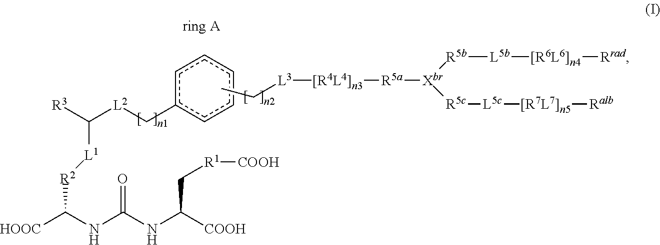
wherein:
R¹ is —R^{1a}R^{1b}—, wherein R^{1a} is absent, —CH₂—, —O—, or —S—, and R^{1b} is —CH₂— or —CHF—;
R² is —(CH₂)₃—O—, —(CH₂)₃—, —(CH₂)₄—, —CH₂—O—(CH₂)₂—, or —CH₂—S—(CH₂)₂—;
L¹ is —S—, —N(R^{L1a})—C(O)—, —C(O)—N(R^{L1a})—, —NH—C(O)—NH—, —NH—C(S)—NH—, 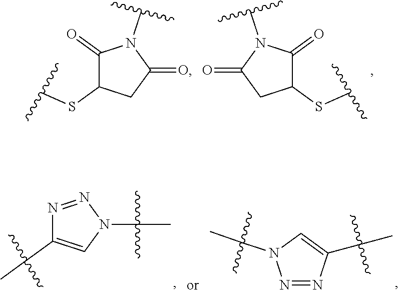
wherein R^{L1a} is H, methyl, ethyl or a benzyl group with 0-4 substituents independently selected from halogen, OMe, or SMe;
R³ is: 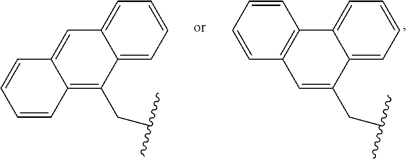 [SMILES: *Cc1c2ccccc2cc2ccccc12 ; *Cc1cc2ccccc2c2ccccc12]
substituted with 0-4 substituents independently selected from C₁-C₄ alkyl, halogen, OMe, SMe, NH₂, NO₂, CN, or OH, and wherein 0-4 ring carbons are replaced with nitrogen;
L² is —S—, —N(R^{L2a})—C(O)—, —C(O)—N(R^{L2a})—, —NH—C(O)—NH—, —NH—C(S)—NH—, 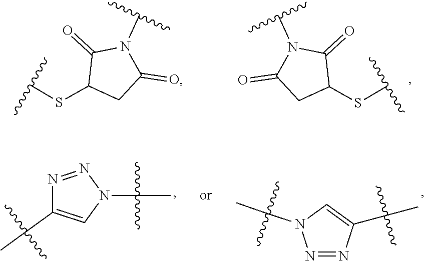
wherein R^{L2a} is H, methyl, or ethyl;
each of n1 and n2 is 0-2;
ring A has 0-3 double bonds, and is bonded at meta or para position;
each of L³, L⁴, L^{5b}, L^{5c}, L⁶, and L⁷ is independently —S—, —N(R^{L3a})—C(O)—, —C(O)—N(R^{L3a})—,
—NH—C(O)—NH—, —NH—C(S)—NH—, 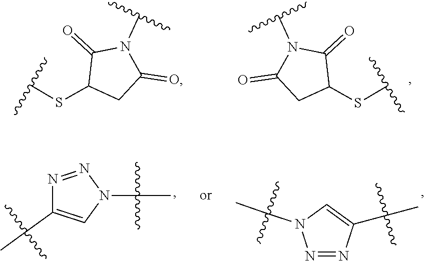
wherein each R^{L3a} is independently H, methyl, or ethyl;
each of n3, n4, and n5 is independently 0-4;
each R⁴ is independently a linear, branched, and/or cyclic Cₙ₆ alkylenyl, alkenylenyl and/or alkynylenyl, wherein each n6 is independently 1-20, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid; and
R^{5a}—X^{br}(R^{5c})—R^{5b} forms a linear, branched, and/or cyclic Cₙ₇ alkylenyl, alkenylenyl and/or alkynylenyl, wherein n7 is 1-20, wherein any carbon bonded to two other carbons is optionally independently replaced by N, S, or O, wherein carbons are optionally independently substituted with oxo, hydroxyl, sulfhydryl, amine, amide, urea, halogen, guanidino, carboxylic acid, sulfonic acid, sulfinic acid, or phosphoric acid, wherein one, two, or three of R^{5a}, R^{5b}, and R^{5c} are optionally absent, wherein X^{br} is CH or N and wherein X^{br} is separated from ring A by at least 4 atoms;
each R⁶ and each R⁷ is independently a linear, branched, and/or cyclic Cₙ₈ alkylenyl, alkenylenyl and/or alkynylenyl, wherein each n8 is independently 1-20, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid;
R^{rad} is a radiometal chelator, optionally bound by a radiometal, wherein R^{rad} is separated from ring A by at least 7 atoms; and
R^{alb} is an albumin binder, wherein the albumin binder is:
—(CH₂)ₙ₉—CH₃ wherein n9 is 8-20;
—(CH₂)ₙ₁₀—C(O)OH wherein n10 is 8-20; or 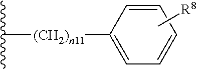
wherein n11 is 1-4 and R⁸ is I, Br, F, Cl, H, OH, OCH₃, NH₂, NO₂, or CH₃; and
R^{alb} is separated from ring A by at least 7 atoms.

2. The compound of claim 1, wherein R^{alb} is separated from ring A by 7-18 atoms, and R^{rad} is separated from ring A by 7-18 atoms.

3. The compound of claim 1 or 2, wherein R^{1a} is —CH₂—, —O—, or —S—, optionally wherein R¹ is —(CH₂)₂—.

4. The compound of any one of claims 1 to 3, wherein R^{alb} is 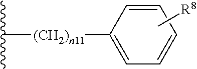
wherein n11 is 1-4 and R⁸ is OCH₃ or NO₂.

5. The compound of claim 4, wherein n11 is 3.

6. The compound of any one of claims 1 to 5, wherein R² is —(CH₂)₄—.

7. The compound of any one of claims 1 to 6, wherein L¹ is —N(R^{L1a})—C(O)— or —C(O)—N(R^{L1a})— wherein L^{1a} is H or methyl, optionally wherein L¹ is —NHC(O)—.

8. The compound of any one of claims 1 to 7, wherein R³ is:

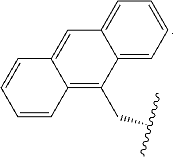

9. The compound of any one of claims 1 to 8, wherein L² is —N(R^{L2a})—C(O)— or —C(O)—N(R^{L2a})—, wherein R^{L2a} is H or methyl, optionally wherein L² is —NHC(O)—.

10. The compound of any one of claims 1 to 9, wherein n1 is 0.

11. The compound of any one of claims 1 to 10, wherein ring A has 0 double bonds and is bonded at para position.

12. The compound of claim 11, wherein ring A is

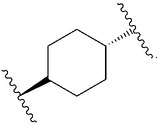

13. The compound of any one of claims 1 to 12, wherein n2 is 0 or 1.

14. The compound of any one of claims 1 to 13, wherein each L³ is independently —N(R^{L3a})—, C(O)— or —C(O)—N(R^{L3a})— wherein each R^{L3a} is independently H or methyl, optionally wherein each L³ is —NHC(O)—.

15. The compound of any one of claims 1 to 14, wherein each L⁴ is independently —N(R^{L4a})—, C(O)— or —C(O)—N(R^{L4a})— wherein each R^{L4a} is independently H or methyl, optionally wherein each L⁴ is —NHC(O)—.

16. The compound of any one of claims 1 to 15, wherein R⁴ is methylene.

17. The compound of any one of claims 1 to 16, wherein n3 is 1.

18. The compound of any one of claims 1 to 17, wherein R^{5a} is absent.

19. The compound of any one of claims 1 to 13, wherein the compound has Formula II or is a salt or a solvate of Formula II:

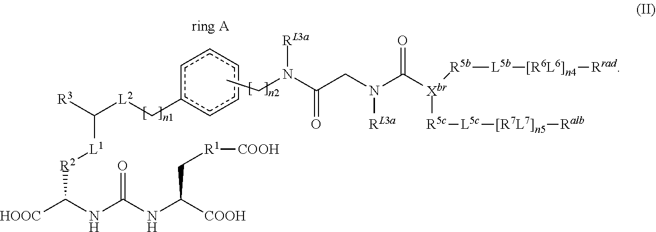

20. The compound of any one of claims 1 to 19, wherein X^{br} is CH.

21. The compound of any one of claims 1 to 20, wherein R^{5b} is absent or —(CH₂)₁₋₄—.

22. The compound of any one of claims 1 to 21, wherein R^{5c} is absent or —(CH₂)₁₋₄—.

23. The compound of any one of claims 1 to 22, wherein L^{5b} is —N(R^{L5b})—C(O)— or —C(O)—N(R^{L5b})— wherein R^{L5b} is H or methyl, optionally wherein L^{5b} is —NHC(O)—.

24. The compound of any one of claims 1 to 23, wherein L^{5c} is —N(R^{L5c})—C(O)— or —(O)—N(R^{L5c})— wherein R^{L5c} is H or methyl, optionally wherein L^{5c} is —NHC(O)—.

25. The compound of any one of claims 1 to 24, wherein each R⁶ is —(CH₂)₁₋₄—.

26. The compound of any one of claims 1 to 25, wherein each L⁶ is independently —N(R^{L6a})—, C(O)— or —C(O)—N(R^{L6a})— wherein each R^{L6a} is independently H or methyl, optionally wherein each L⁶ is —NHC(O)—.

27. The compound of any one of claims 1 to 26, wherein n4 is 0 or 1.

28. The compound of any one of claims 1 to 27, wherein each R⁷ is —(CH₂)₁₋₄—.

29. The compound of any one of claims 1 to 28, wherein each L⁷ is independently —N(R^{L7a})—, C(O)— or —C(O)—N(R^{L7a})— wherein each R^{L7a} is independently H or methyl, optionally wherein each L⁷ is —NHC(O)—.

30. The compound of any one of claims 1 to 29, wherein n5 is 0 or 1.

31. The compound of any one of claims 1 to 30, wherein X^{br} is separated from ring A by at least 5 atoms, optionally at least 6 atoms.

32. The compound of claim 1 or claim 19, wherein:
R¹ is —R^{1a}R^{1b}—, wherein R^{1a} is absent or —CH₂—, and R^{1b} is —CH₂— or —CHF—;
R² is —(CH₂)₃—O—, —(CH₂)₃—, —(CH₂)₄—, or —CH₂—O—(CH₂)₂—;
L¹ is —N(R^{L1a})—C(O)—, —C(O)—N(R^{L1a})—, or —NH—C(O)—NH—, wherein R^{L1a} is H or methyl;
R³ is: 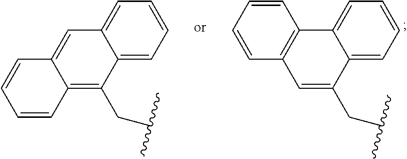 [SMILES: *Cc1c2ccccc2cc2ccccc12 ; *Cc1cc2ccccc2c2ccccc12]
L² is —N(R^{L2a})—C(O)—, —C(O)—N(R^{L2a})—, —NH—C(O)—NH—, wherein R^{L2a} is H, or methyl;
each of n1 and n2 is 0-2;
ring A has 0 double bond, and is bonded at meta or para position;
each of L³, L⁴, L^{5b}, L^{5c}, L⁶, and L⁷ is independently —N(R^{L3a})—C(O)—, —C(O)—N(R^{L3a})—, or —NH—C(O)—NH—, wherein each R^{L3a} is independently H, methyl, or ethyl;
each of n3, n4, and n5 is independently 0-4;
each R⁴ is independently a linear C₁-C₁₀ alkylenyl;
R^{5a}—X^{br}(R^{5c})—R^{5b} forms a linear, branched, and/or cyclic C₁-C₂₀ alkylenyl, alkenylenyl and/or alkynylenyl, wherein any carbon bonded to two other carbons is optionally independently replaced by N, S, or O, wherein carbons are optionally independently substituted with oxo, hydroxyl, sulfhydryl, amine, amide, urea, halogen, guanidino, carboxylic acid, sulfonic acid, sulfinic acid, or phosphoric acid, wherein one, two, or three of R^{5a}, R^{5b}, and R^{5c} are optionally absent, wherein X^{br} is N or CH, and wherein X^{br} is separated from ring A by at least 4 atoms;
each R⁶ and each R⁷ is independently a linear, branched, and/or cyclic C₁-C₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, amine, amide, urea, halogen, guanidino, carboxylic acid, sulfonic acid, sulfinic acid, or phosphoric acid;
R^{rad} is a radiometal chelator, optionally bound by a radiometal, wherein R^{rad} is separated from ring A by at least 7 atoms; and
R^{alb} is: 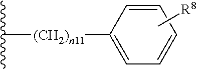
wherein n11 is 1-4 and R⁸ is I, Br, F, Cl, H, OH, OCH₃, NH₂, NO₂, or CH₃.

33. The compound of claim 32, wherein:
R² is —(CH₂)₃—, or —(CH₂)₄—;
L¹ is —N(R^{L1a})—C(O)—, or —C(O)—N(R^{L1a})—, wherein R^{L1a} is H or methyl;
R³ is: 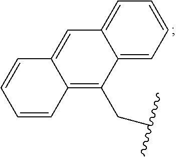 [SMILES: *Cc1c2ccccc2cc2ccccc12]
L² is —N(R^{L2a})—C(O)—, or —C(O)—N(R^{L2a})—, wherein R^{L2a} is H, or methyl;
each of n1 and n2 is 0-1;
each of L³, L⁴, L^{5b}, L^{5c}, L⁶, and L⁷ is independently —N(R^{L3a})—C(O)—, or —C(O)—N(R^{L3a})—,
wherein each R^{L3a} is independently H or methyl;
each R⁴ is independently a linear C₁-C₁₀ alkylenyl;
R^{5a}—X^{br}(R^{5c})—R^{5b} forms a linear, branched, and/or cyclic C₁-C₁₀ alkylenyl, wherein any carbon bonded to two other carbons is optionally independently replaced by O, wherein carbons are optionally independently substituted with oxo, hydroxyl, amine, amide, or carboxylic acid, wherein one, two, or three of R^{5a}, R^{5b}, and R^{5c} are optionally absent, wherein X^{br} is N or CH and wherein X^{br} is separated from ring A by at least 4 atoms;
each R⁶ and each R⁷ is independently a linear, branched, and/or cyclic C₁-C₁₀ alkylenyl, wherein any carbon bonded to two other carbons is optionally independently replaced by N, S, or O, and carbons are optionally independently substituted with oxo, hydroxyl, amine, amide, urea, carboxylic acid; and
R^{alb} is: 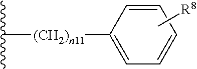
wherein n11 is 1-4 and R⁸ is OCH₃, or NO₂.

34. The compound of claim 32 or 33, wherein L³ is —NHC(O)—.

35. The compound of any one of claims 32-34, wherein L⁴ is —NHC(O)—.

36. The compound of any one of claims 32-35, wherein R⁴ is methylene.

37. The compound of any one of claims 32-36, wherein n3 is 1-4.

38. The compound of claim 37, wherein n3 is 1-2.

39. The compound of claim 37, wherein n3 is 1.

40. The compound of any one of claims 32-39, wherein n2 is 0-1.

41. The compound of claim 40, wherein n2 is 1.

42. The compound of any one of claims 32-41, wherein R^{5a} is absent.

43. The compound of any one of claims 32-41, wherein R^{5a} is methylene.

44. The compound of any one of claims 1, 19, 32 or 33, wherein
ring A is 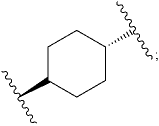
R² is —(CH₂)₄—;
R³ is: 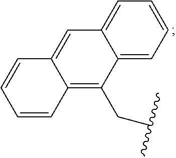 [SMILES: *Cc1c2ccccc2cc2ccccc12]
n1 is 0;
L¹ is —N(R^{L1a})—C(O)—, or —C(O)—N(R^{L1a})—, wherein R^{L1a} is H;
L² is —N(R^{L2a})—C(O)—, or —C(O)—N(R^{L2a})—, wherein R^{L2a} is H; and
R¹ is —R^{1a}R^{1b}—, wherein R^{1a} is —CH₂—, and R^{1b} is —CH₂— or —CHF—.

45. The compound of claim 44, wherein
R^{5c} is absent;
L^{5c} is —N(R^{L3a})—C(O)— or —C(O)—N(R^{L3a})—, wherein R^{L3a} is H;
R⁷ is methylene;
L⁷ is —N(R^{L3a})—C(O)— or —C(O)—N(R^{L3a})—, wherein R^{L3a} is H;
n5 is 1; and
R^{alb} is: 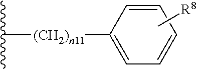
wherein n11 is 3 and R⁸ is OCH₃, or NO₂.

46. The compound of claim 44 or 45, wherein
n2 is 0 or 1;
L³ is —N(R^{L3a})—C(O)—, or —C(O)—N(R^{L3a})—, wherein R^{L3a} is H;
R⁴ is methylene;
L⁴ is —N(R^{L3a})—C(O)—, or —C(O)—N(R^{L3a})—, wherein R^{L3a} is H;
n3 is 1;
R^{5a} is absent; and
X^{br} is CH;

47. The compound of any one of claims 44-46, wherein
R^{5b} is a linear C₁-C₆ alkylenyl;
L^{5b} is —N(R^{L3a})—C(O)— or —C(O)—N(R^{L3a})—, wherein R^{L3a} is H;
R⁶ is methylene;
L⁶ is —N(R^{L3a})—C(O)— or —C(O)—N(R^{L3a})—, wherein R^{L3a} is H; and
n4 is 0-2.

48. The compound of any one of claims 1-47, wherein R^{rad} is selected from Table 2; and wherein R^{rad} is optionally bound to a radiometal.

49. The compound of any one of claims 1-48, wherein R^{rad} is DOTA, H₂macropa, H₄ py4 pa, H₄Pypa, or CROWN, wherein R^{rad} is optionally bound to a radiometal.

50. The compound of claim 49, wherein R^{rad} is DOTA.

51. The compound of any one of claims 1 to 50, wherein R^{rad} is bound by a therapeutic radiometal, optionally wherein the therapeutic radiometal is ¹⁶⁵Er, ²¹²Bi, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ²²⁴Ra, ²²⁷Th, ²²³Ra, ⁶⁴Cu, or ⁶⁷Cu.

52. A compound selected from CCZ02009, CCZ02017, CCZ02008, CCZ02025, CCZ02024, CCZ02015, CCZ02019, CCZ02012, CCZ02005, CCZ02021, CCZ02022, CCZ02059, CCZ02060, CCZ02034, CCZ02061, or CCZ02013, wherein the compound is optionally bound to a radiometal.

53. A compound selected from CCZ02005, CCZ02021, CCZ02022, CCZ02059, CCZ02060, CCZ02034, CCZ02061, wherein the compound is optionally bound to a radiometal.

54. A compound selected from CCZ02009, CCZ02017, CCZ02008, CCZ02025, CCZ02024, CCZ02015, or CCZ02019, wherein the compound is optionally bound to a radiometal.

55. The compound of any one of claims 1-54, wherein the radiometal is ¹⁶⁵Er, ²¹²Bi, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²¹²Pb, ⁴⁷Sc, ⁹⁰Y, ²²⁵Ac, ¹¹⁷mSn, ¹⁵³Sm, ¹⁴⁹Tb, ¹⁶¹Tb, ²²⁴Ra, ²²⁷Th, ²²³Ra, ⁶⁴Cu, or ⁶⁷Cu.

56. The compound of claim 55, wherein the radiometal is ¹⁷⁷Lu or ²²⁵Ac.

57. A pharmaceutical composition comprising a compound of any one of claims 1-56 and one or more pharmaceutically acceptable excipients.

58. The compound of any one of claims 1-56 or the pharmaceutical composition of claim 57, for use in treatment of a prostate-specific membrane antigen (PSMA) expressing tumor in a subject.

59. The compound of claim 58, wherein the prostate-specific membrane antigen (PSMA) expressing tumor relates to prostate cancer.

60. The compound of claim 58 or 59, wherein the subject is human.
